(19) United States
(12) Patent Application Publication
(10) Pub. No.: US 2024/0299032 A1
(43) Pub. Date: Sep. 12, 2024

(54) INCISION CLOSURE AND TUBE HOLDER APPARATUS AND METHOD

(71) Applicant: ROYAL COLUMBIAN HOSPITAL FOUNDATION, New Westminster (CA)

(72) Inventors: Ali ABDALVAND, Coquitlam (CA); Nigel Bruce Halsted, Surrey (CA); Thomas Lloyd Bellaire, Vancouver (CA)

(21) Appl. No.: 18/283,775

(22) PCT Filed: Dec. 21, 2021
(86) PCT No.: PCT/CA2021/051862

§ 371 Date: Sep. 22, 2023

Related U.S. Application Data
(60) Provisional application No. 63/165,669, filed on Mar. 24, 2021.

(51) Int. Cl. A61B 17/08; A61B 17/10; A61M 27/00

(52) U.S. Cl. CPC A61B 17/08; A61B 17/10; A61M 27/00; A61B 2017/081; A61M 2210/10

(57) ABSTRACT

An incision closure apparatus and method for securely and adjustably holding a tube passing through the incision, such as a chest tube. In some embodiments the apparatus comprises a frame having a first side and a second side; a handle assembly pivotably coupled to the frame, the assembly comprising a pair of handles disposed between the first side and second side of the frame, wherein each of the handles is pivotably coupled to the frame and is movable relative to the frame between non-deployed and deployed positions; a needle array positionable at a lower end of each of the handles; and a tube holder located adjacent one of the first side and the second side of the frame for releasably securing the tube. The apparatus is adjustable to alter the placement of the tube and to vary the pressure applied to the tissue at the site of the closed incision by adjusting the spacing between the pair of handles.

### CROSS-REFERENCE TO RELATED APPLICATIONS

This application claims priority from U.S. Application No. 63/165,669 filed 24 Mar. 2021 and entitled INCISION CLOSURE AND TUBE HOLDER APPARATUS AND METHOD which is hereby incorporated herein by reference for all purposes. For purposes of the United States of America, this application claims the benefit under 35 U.S.C. § 119 of U.S. application No. 63/165,669 filed 24 Mar. 2021 and entitled INCISION CLOSURE AND TUBE HOLDER APPARATUS AND METHOD.

### TECHNICAL FIELD

This application relates to an apparatus and method for closing an incision formed in the bodily tissue of a patient and for securely holding a tube inserted through the incision. In some embodiments the apparatus and method relates to stabilization and securement of a chest tube inserted through an incision formed in the chest wall of the patient.

### BACKGROUND

A chest tube is a hollow, flexible tube placed into the chest of a patient, for example to drain fluid, blood and/or air from the pleural cavity. Chest tubes are commonly used, for example, to treat patients having collapsed lungs or internal fluid buildup. In order to insert a chest tube, an incision is made in the chest wall of the patient. After the chest tube is placed at the desired location, the incision is ordinarily closed and the chest tube is secured with surgical sutures. This requires considerable surgical skill since the sutures must be sufficiently tight to ensure that the tube does not move or fall out of the body cavity entirely. Many physicians and other medical personnel are not sufficiently familiar with suture techniques to close a chest tube incision quickly and reliably. For example, emergency physicians or other care providers working in remote locations, sometimes without any back-up medical support or assistance, may not be regularly called upon to insert chest tubes and hence may not be practiced in the optimum procedures for tube placement and securement. Even if the attending physician is familiar within the preferred techniques, the suturing procedure is time-consuming and cannot be easily delegated to non-medical staff.

Various devices are known in the prior art for holding and securing a chest tube and for sealing surgical incisions or other wounds in the vicinity of a chest tube. In some cases such devices can be deployed by first-responders or other care providers lacking specialized medical training. For example, US patent publication No. 2014/0309687 to Atkinson et al. published 16 Oct. 2014 relates to a wound clamp that can also anchor a chest tube or other medical instrument in some embodiments. The Atkinson et al. clamp is particularly designed for closing a wound in emergency situations, such as during military operations or civilian disaster situations. To that end, the Atkinson wound clamp is intended to stabilize a patient shortly after injury pending transport to a hospital for emergency surgery or other necessary medical procedures. The Atkinson et al. clamp is not particularly adapted for extended use or post-installation adjustment.

Various other devices are known in the prior art for securing chest tubes, catheters and the like. Some prior art devices employ adhesives to temporarily secure the tube to the patient. However, in many cases such adhesives are time-consuming to deploy and do not provide a reliable means for anchoring a tube in the desired orientation.

In the treatment of some medical conditions it may be desirable to leave chest tubes in place for extended periods of time. For example, some lung cancers can cause fluid to reaccumulate in the pleural cavity and physicians may elect to leave a chest tube in a patient for an extended treatment period. During that time the patient may be frequently moving between supine, sitting and standing positions. It is important that the chest tube remain at the optimum placement and the incision remains safely sealed. As will be appreciated by a person skilled in the art, if a chest tube remains in place for an extended period of time there is a heightened risk of infection or other potentially serious side effects. Accordingly, there is a need for an incision closure apparatus and method which is adjustable to vary both the precise placement of the chest tube and the degree of tissue pressure at the site of the closed incision to reduce the risk of infection, tissue necrosis or other deleterious side effects. To that end, it is desirable that the apparatus does not obscure visualization of the incision by the attending physician or other care provider during deployment of the apparatus and post-closure recovery.

The foregoing examples of the related art and limitations related thereto are intended to be illustrative and not exclusive. Other limitations of the related art will become apparent to those of skill in the art upon a reading of the specification and a study of the drawings.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

One aspect of the invention provides an apparatus for closing an incision formed in a patient and for releasably securing a tube passing through the incision. The apparatus comprises a frame having a first side and a second side; a handle assembly pivotably coupled to the frame, the assembly comprising a pair of handles at least partially disposed between the first side and the second side; a needle array positionable at a first end of each of the handles for penetrating tissue of the patient adjacent to the incision; and a tube holder located adjacent one of the first side and the second side for securing the tube.

In some embodiments, each of the handles is movable relative to the frame between a disengaged position wherein the needle array does not penetrate the tissue of the patient and an engaged position wherein the needle array penetrates the tissue of the patient. The needle array may be mounted on or removably connectable to a handle. In some particular embodiments, each of the handles comprises a housing and a needle cartridge comprising the needle array, wherein the cartridge is movable in the housing between the disengaged and the engaged positions.

In some embodiments, the handle assembly is movable between an inserted position wherein the needle array of each of the handles penetrates the tissue of the patient and an everted position wherein the tissue of the patient engaged by the handles is everted. In some embodiments the needle array of a first one of the handles engages the tissue on a first side of the incision and the needle array of a second one of the handles engages the tissue on a second side of the incision, wherein the handle assembly is configured for bringing everted tissue on the first side and the second side together to close the incision. The handle assembly may be pivotably movable between the inserted position and the everted position and may be releasably lockable in the everted position. In some embodiments, the distance between the handles is adjustable in the everted position to adjust the compressive force applied to the tissue on either side of the incision in contact when the incision is closed. In some embodiments each of the handles may comprise laterally extending pivot pins each movable within a corresponding guide slot for pivotally coupling each of the handles to the frame.

In some embodiments, the incision has a length between a first end and a second end thereof, and the apparatus is positionable on the patient with the incision extending between the handles. In some embodiments the tube holder is positionable overlying the first end or the second end of the incision and is configured to securely receive a chest tube extending through an end portion of the incision. The handles may comprise a first handle and a second handle defining a space therebetween, wherein the space is generally aligned with a transverse centreline of the frame extending between the first side and the second side. In some embodiments, the tube guide is a U-shaped holder having a central portion aligned with the transverse centreline. The apparatus may be positionable on the patient such that the length of the incision is generally aligned with the transverse centreline of the frame.

In some embodiments the needle array comprises a plurality of spaced-apart needles. In some embodiments, at least some of the needles are oriented at a non-vertical angle differing from the other needles. For example, one or more needles proximal to the tube holder may be oriented at an angle and/or have a length differing from other needles of the array.

Another aspect of invention comprises a method of closing an incision formed in a patient, the incision having a length extending between a first end and a second end thereof, the method comprising providing an apparatus as described above; positioning the apparatus overlying the incision such that the incision is aligned between the handles; penetrating the needle array of each of the handles into the tissue of the patient adjacent to the incision; moving the handles relative to the frame to evert the tissue adjacent to the incision; and bringing everted tissue on either side of the incision into contact to close the incision. The method may further comprise releasably securing a tube passing through the incision within a tube holder.

Another aspect of the invention comprises making an incision in a chest wall of the patient; inserting the chest tube through the incision and placing the chest tube at one end of the incision; providing an apparatus as described above; positioning the apparatus such that the incision is aligned between the handles and the chest tube is received within the tube holder; releasably securing the chest tube in the tube holder; moving the needle array relative to the frame from a disengaged position to an engaged position to cause the needles to penetrate tissue adjacent the incision; and moving the handles relative to the frame in the engaged position to evert the tissue adjacent to the incision and bring the everted tissue in contact to close the incision.

In another aspect of the invention the apparatus may be used to close an incision without concurrently holding a tube. In one embodiment a method of closing an incision opening formed in tissue of a patient is provided comprising: providing an incision closure apparatus comprising a frame and a handle assembly movably coupled to the frame, wherein the handle assembly comprises a first handle comprising a first needle array and a second handle spaced-apart from the first handle and comprising a second needle array; positioning the incision closure apparatus on the patient with the incision opening disposed between the first and second handles; inserting the first needle array into the tissue adjacent a first side of the incision opening and inserting the second needle array into the tissue adjacent a second side of the incision opening; moving the handles relative to the frame to evert the tissue engaged by the first and second needle arrays; and closing the incision opening by moving the handles toward one another to bring the everted tissue in contact in a closed position. In the closed position the first and second handles may be releasably locked to the frame.

In addition to the exemplary aspects and embodiments described above, further aspects and embodiments will become apparent by reference to the drawings and by study of the following detailed descriptions.

### BRIEF DESCRIPTION OF THE DRAWINGS

Exemplary embodiments are illustrated in referenced figures of the drawings. It is intended that the embodiments and figures disclosed herein are to be considered illustrative rather than restrictive.

FIG. 1 is an isometric view of a patient in a supine position with an incision formed in the patient's chest wall and showing one embodiment of an apparatus in accordance with the invention in a deployed configuration to secure a chest tube extending through the incision.

FIG. 2 is an isometric view of the apparatus of FIG. 1 in an initial, non-deployed position with the needle cartridges disengaged and the needle arrays retracted within the handle assembly.

FIG. 3 is an isometric, partially exploded view of the apparatus of FIG. 2.

FIG. 4 is a first side view of the apparatus of FIG. 2 with a first sidewall distal from the tube holder assembly omitted to more clearly show internal components.

FIG. 5 is a second side view of the apparatus of FIG. 2 with the tube holder assembly and a second sidewall proximal to the tube holder assembly omitted to more clearly show internal components.

FIG. 6 a top, plan view of the apparatus of FIG. 2.

FIG. 7A is an end view of the apparatus of FIG. 2.

FIG. 7B is an end view of the apparatus of FIG. 2 showing the interior of the needle cartridge with some parts of the handle removed for clarity and showing the cartridge in an initial position with the needle array retracted within the handle housing.

FIG. 7C is an end view of the apparatus of FIG. 7B showing the needle cartridge in a depressed position with the needle array extending from the handle housing.

FIG. 8 is an isometric view of the apparatus of FIG. 1 in a partially deployed position with the needle cartridges fully inserted within the handle assembly to extend the needle arrays.

FIG. 9 is a first side view of the apparatus of FIG. 8 with a first sidewall distal from the tube holder assembly omitted to more clearly show internal components.

FIG. 10 is a second side view of the apparatus of FIG. 8 with the tube holder assembly and a second side wall proximal to the tube holder assembly omitted to more clearly show internal components.

FIG. 11 is an end view of the apparatus of FIG. 8.

FIG. 12 is an isometric view of the apparatus of FIG. 1 in a further deployed position with the handle assembly partially pivoted to evert tissue adjacent to the incision.

FIG. 13 is a first side view of the apparatus of FIG. 12 with a first sidewall distal from the tube holder assembly omitted to more clearly show internal components.

FIG. 14 is a second side view of the apparatus of FIG. 12 with the tube holder assembly and a second side wall proximal to the tube holder assembly omitted to more clearly show internal components.

FIG. 15 is a top, plan view of the apparatus of FIG. 12.

FIG. 16 is an end view of the apparatus of FIG. 12.

FIG. 17 is an isometric view of the apparatus of FIG. 1 in a fully deployed position with the handle assembly fully pivoted and secured in a closed position.

FIG. 18 is a first side view of the apparatus of FIG. 17 with a first sidewall distal from the tube holder assembly omitted to more clearly show internal components.

FIG. 19 is a second side view of the apparatus of FIG. 17 with the tube holder assembly and a second side wall proximal to the tube holder assembly omitted to more clearly show internal components.

FIG. 20 is a top, plan view of the apparatus of FIG. 17.

FIG. 21 is an end view of the apparatus of FIG. 17.

FIG. 22 is an isometric view of the apparatus of FIG. 1 showing post-closure adjustment of the handle assembly to vary the distance between the needle arrays.

FIG. 23 is a first side view of the apparatus of FIG. 22 with a first sidewall distal from the tube holder assembly omitted to more clearly show internal components.

FIG. 24 is a second side view of the apparatus of FIG. 22 with the tube holder assembly and a second side wall proximal to the tube holder assembly omitted to more clearly show internal components.

FIG. 25 is a top, plan view of the apparatus of FIG. 22.

FIG. 26 an end view of the apparatus of FIG. 22.

FIG. 27A is an isometric view of the apparatus of FIG. 1 in an initial step of a deployment procedure holding a chest tube at one end of an incision and showing the needle cartridges disengaged.

FIG. 27B a top plan view thereof.

FIG. 28A is an isometric view of the apparatus of FIG. 1 in a further step of a deployment procedure showing the apparatus partially deployed with the needle cartridges engaged and the incision partially closed.

FIG. 28B is a top, plan view thereof.

FIG. 29A is an isometric view of the apparatus of FIG. 1 in a further step of a deployment procedure showing the apparatus fully deployed and the incision substantially closed.

FIG. 29B is a top, plan view thereof.

FIG. 30A is an isometric view of the apparatus of FIG. 29A further adjusted to reduce the spacing between the needle arrays and hence the width of the incision.

FIG. 30B is a top, plan view thereof.

FIG. 31 is an isometric view of another embodiment of the apparatus in a fully deployed configuration.

FIG. 32 is an isometric, partially exploded view of the apparatus of FIG. 31.

FIG. 33 is a first side view of the apparatus of FIG. 31 in an initial, non-deployed position.

FIG. 34 is an enlarged second side view thereof.

FIG. 35 is a top, plan view thereof.

FIG. 36 is an end view thereof.

FIG. 37 is a first side view of the apparatus of FIG. 31 in a partially deployed position with the needle arrays engaged.

FIG. 38 is a second side view thereof.

FIG. 39 is an end view thereof.

FIG. 40 is a first side view of the apparatus of FIG. 31 with the handle assembly pivoted to a partially deployed position.

FIG. 41 is a second side view thereof.

FIG. 42 is a top, plan view thereof.

FIG. 43 is an end view thereof.

FIG. 44 is a first side view of the apparatus of FIG. 31 with the handle assembly further pivoted and inwardly advanced to a fully deployed, closed position.

FIG. 45 is a second side view thereof.

FIG. 46 is a top, plan view thereof.

FIG. 47 is an end view thereof.

### DESCRIPTION

Throughout the following description specific details are set forth in order to provide a more thorough understanding to persons skilled in the art. However, well known elements may not have been shown or described in detail to avoid unnecessarily obscuring the disclosure. Accordingly, the description and drawings are to be regarded in an illustrative, rather than a restrictive, sense.

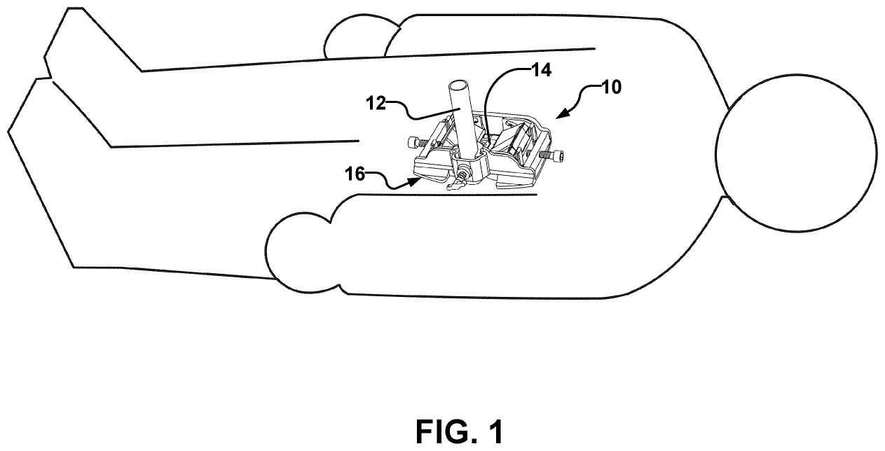

With reference to FIG. 1, this application relates in some embodiments to an apparatus 10 configured to: (1) securely and releasably hold a tube 12 inserted through an incision 14 and (2) close incision 14 after securement of tube 12. As used in this patent application, the term “incision” may refer to a surgically created opening formed in tissue of a patient and any other wound or other temporary opening formed in a patient, for example due to accident or injury.

In some embodiments tube 12 may be a chest tube inserted through a surgical incision 14 formed in a chest wall of a patient. As will be appreciated by those skilled in the art, a chest tube is a hollow, flexible tube placed into the chest of a patient for example to drain fluid, blood and/or air from the pleural cavity or other interior body location. The medical procedure for inserting a chest tube, also sometimes referred to as tube thoracostomy, chest drain or intercostal drain, is a common therapy for treating hemothorax or tension pneumothorax. As discussed in detail below, in some embodiments apparatus 10 is configured to securely and releasably hold tube 12 at one end of incision 14 and to close incision 14 along the length thereof without the need for surgical sutures, adhesives, and/or other medical instruments.

In some alternative embodiments described below, apparatus 10 may comprise an incision closure apparatus which may be used to close an incision without the need for surgical sutures, adhesives, and/or other medical instruments that is not configured to also concurrently hold a tube 12. For example, apparatus 10 may be used to close an incision made for cosmetic purposes or other purposes unrelated to deploying a tube 12.

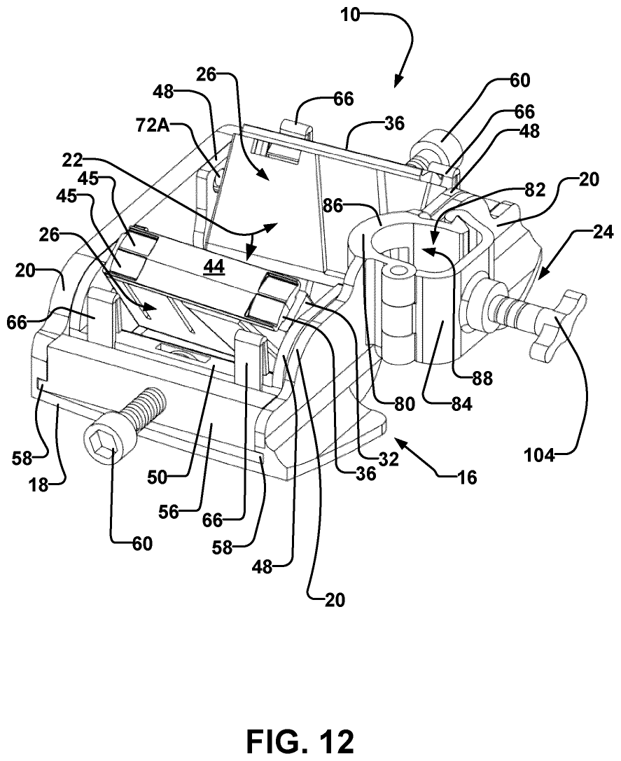

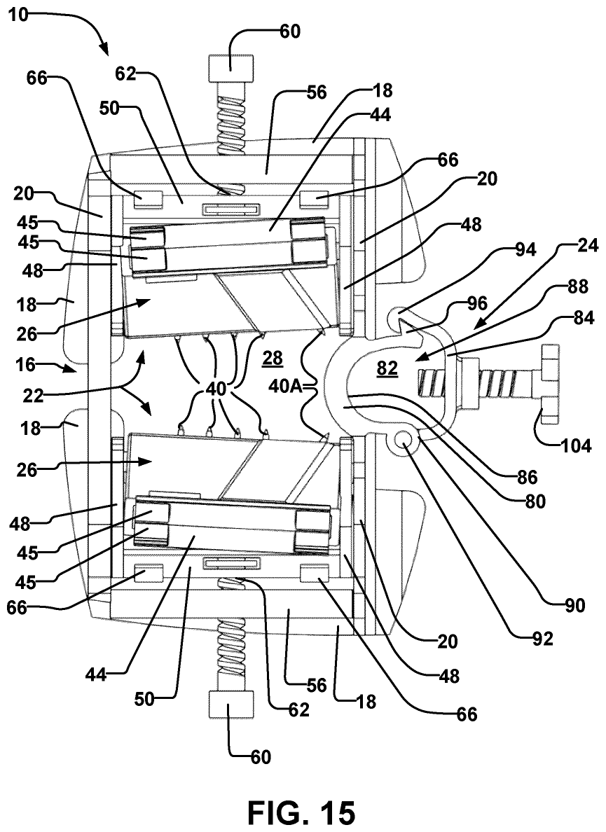

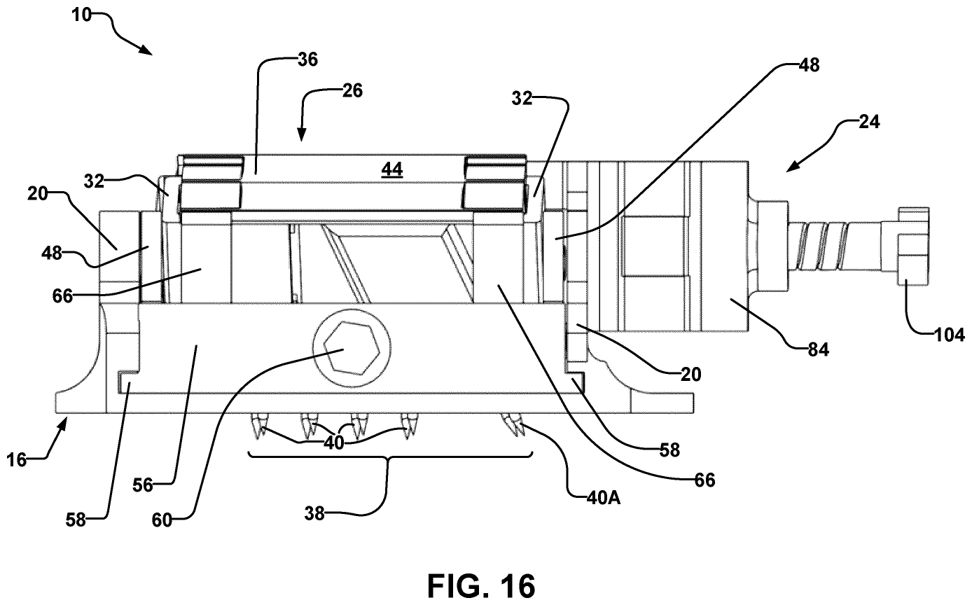

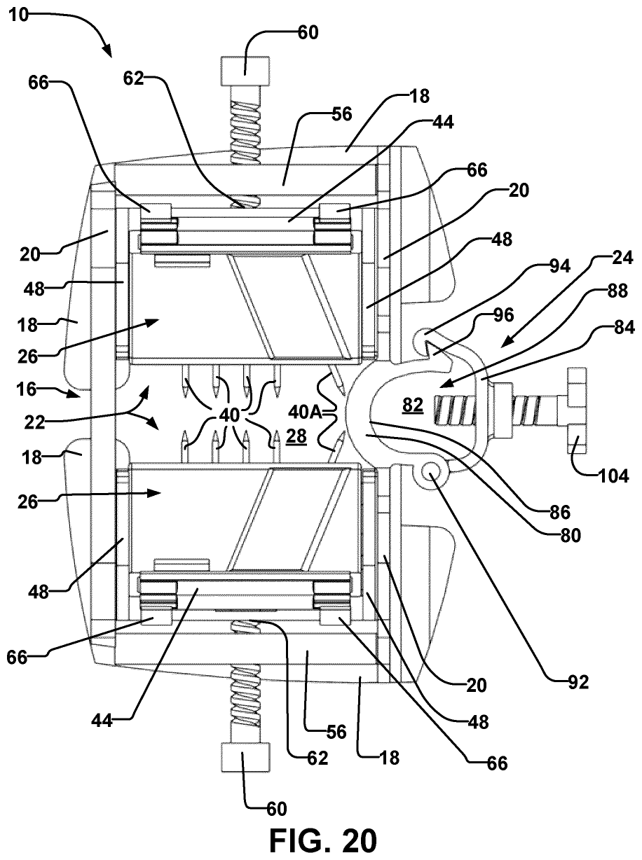

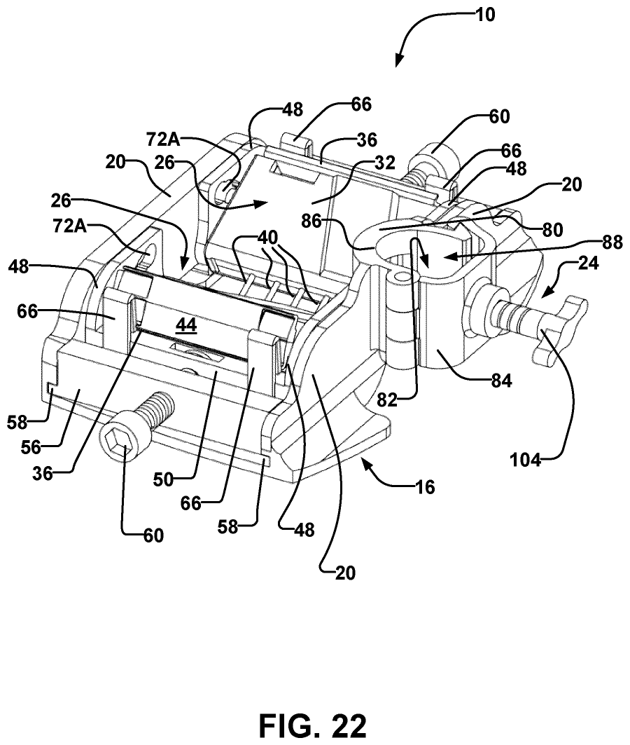

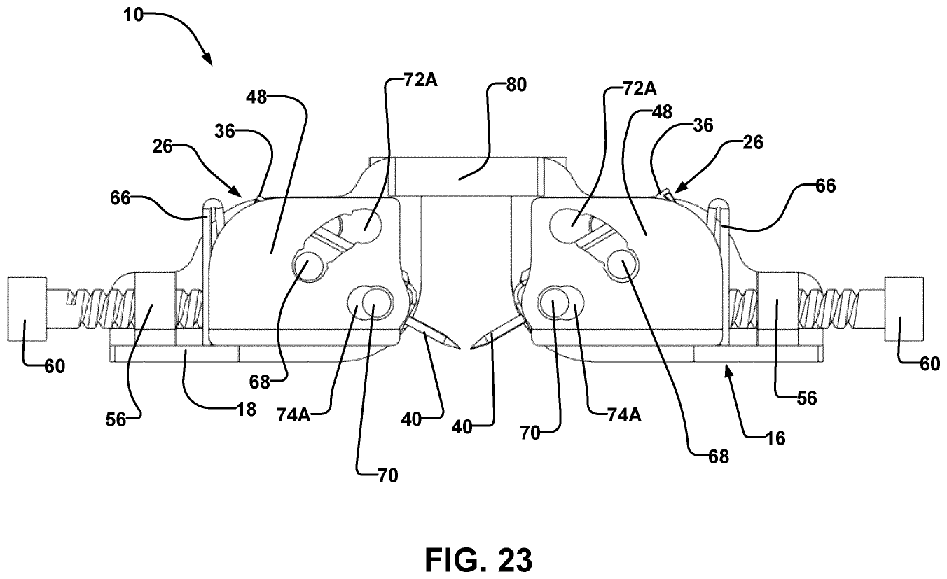

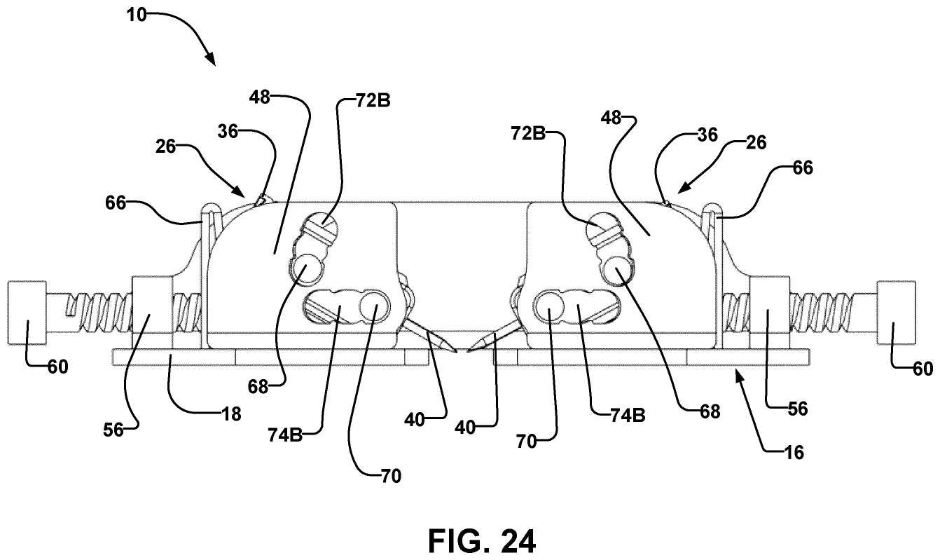

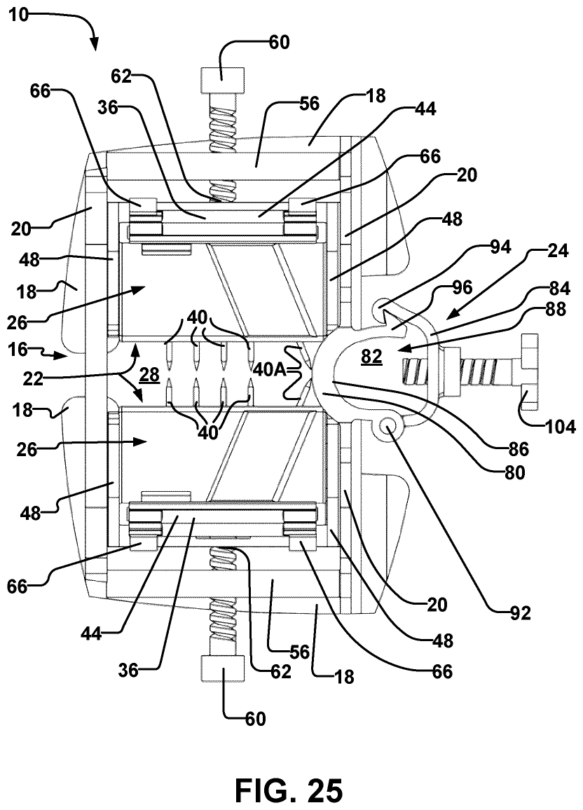

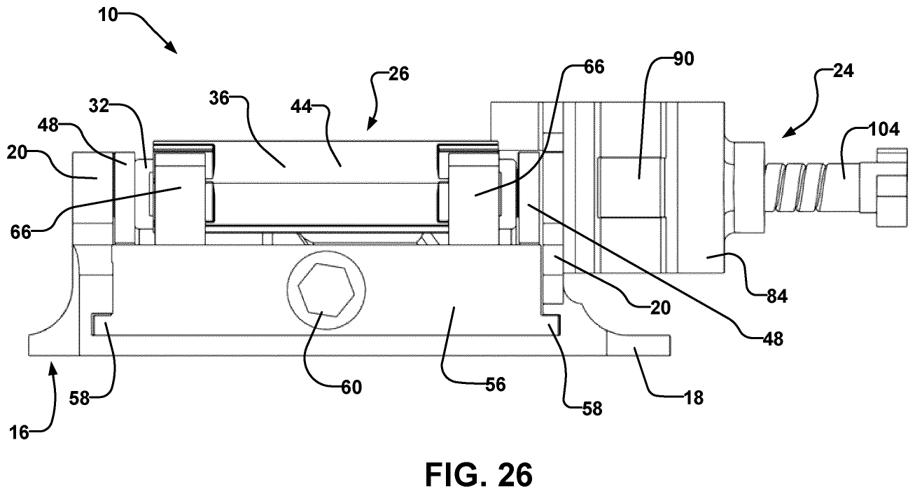

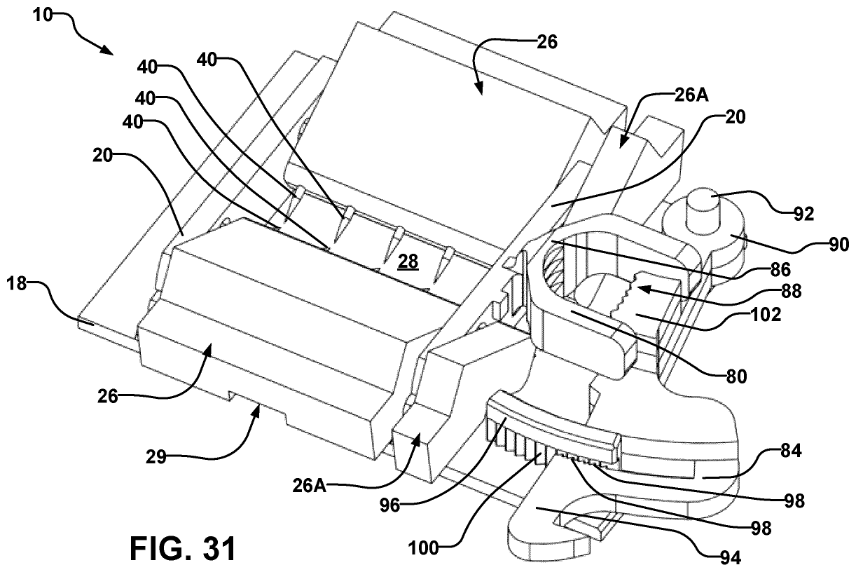

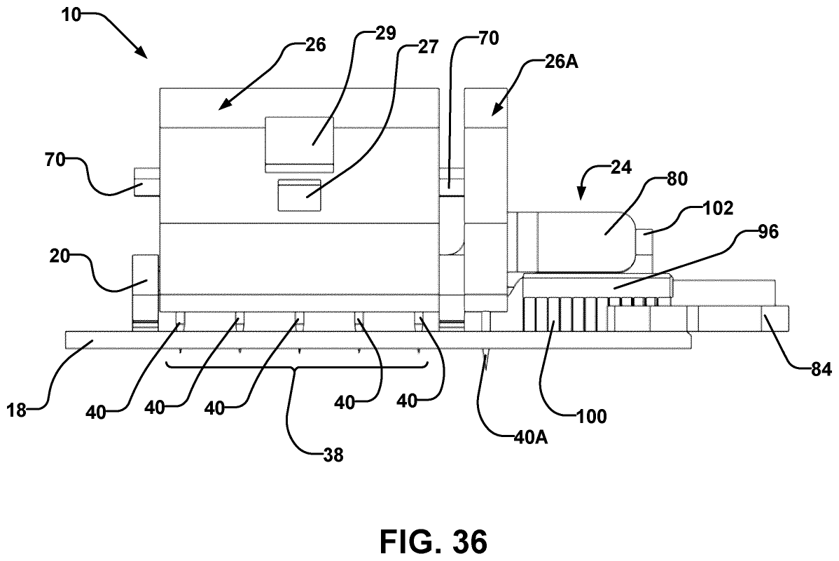

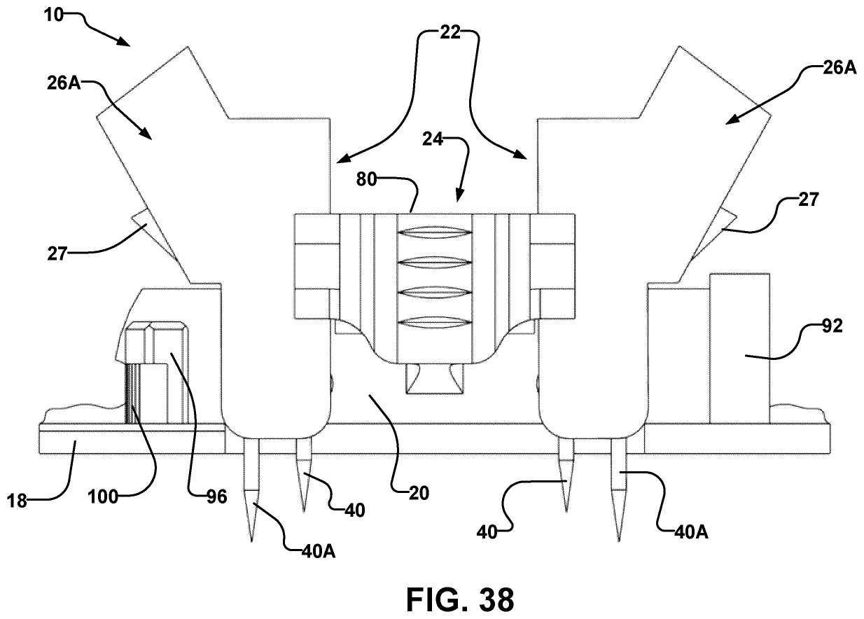

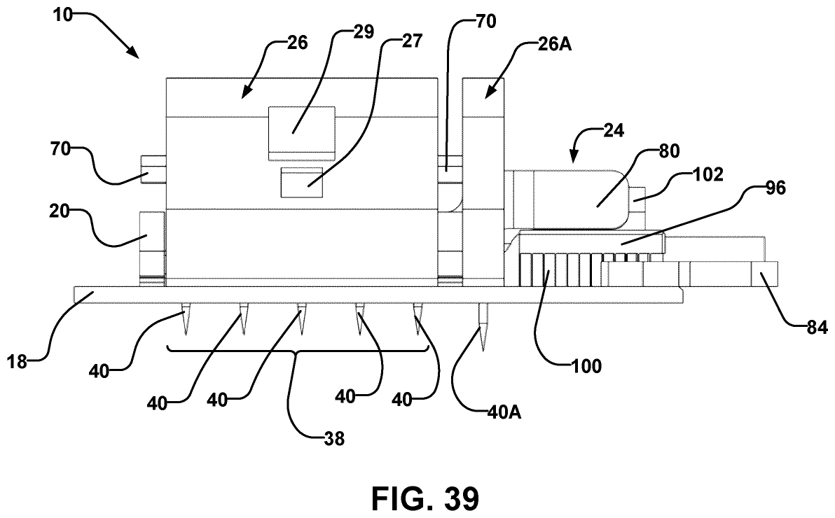

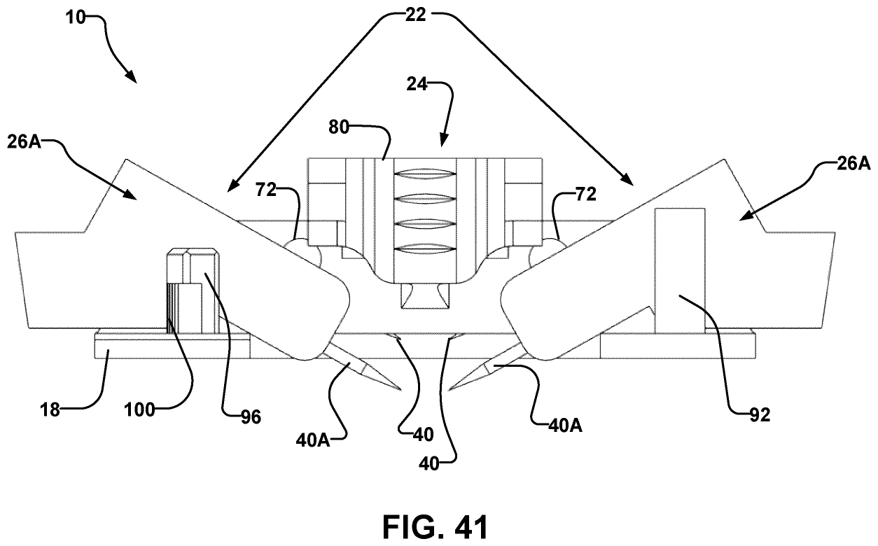

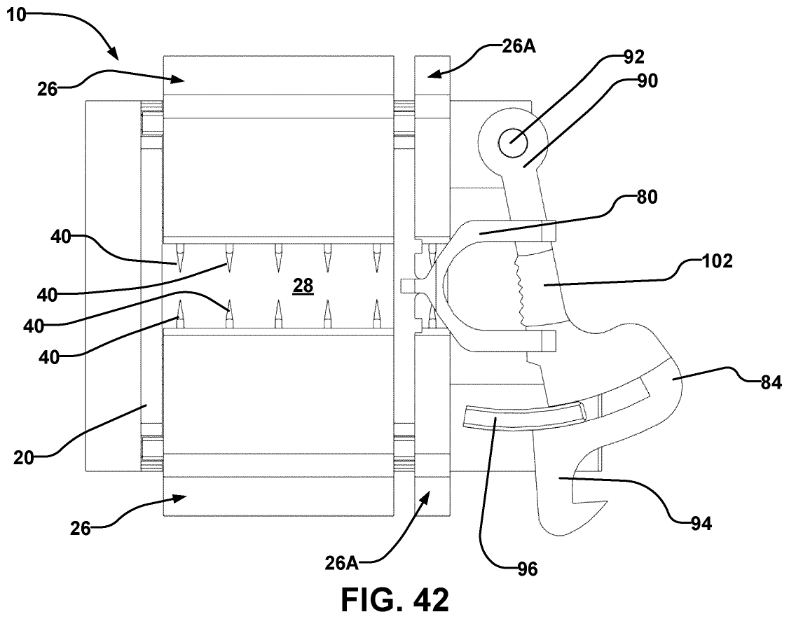

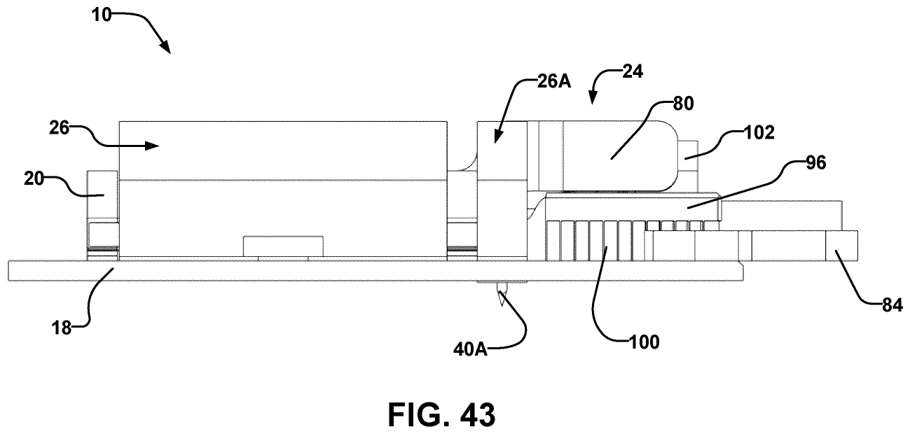

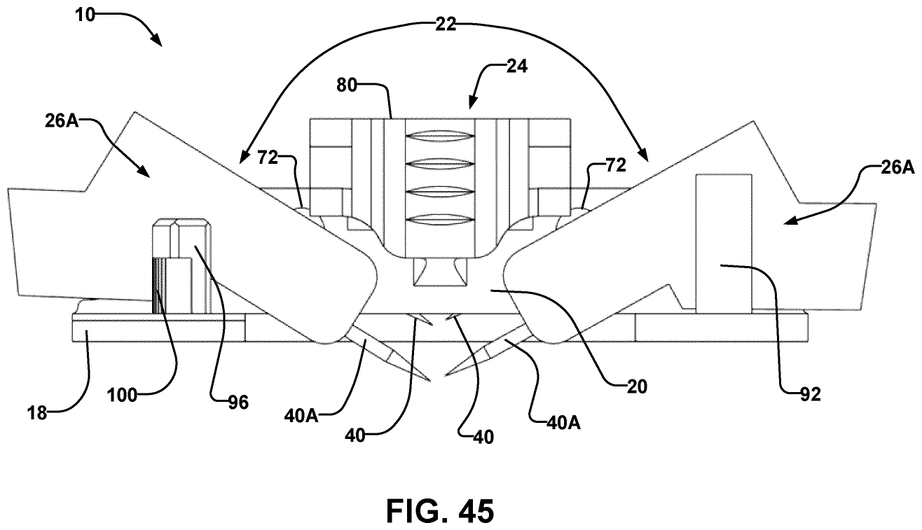

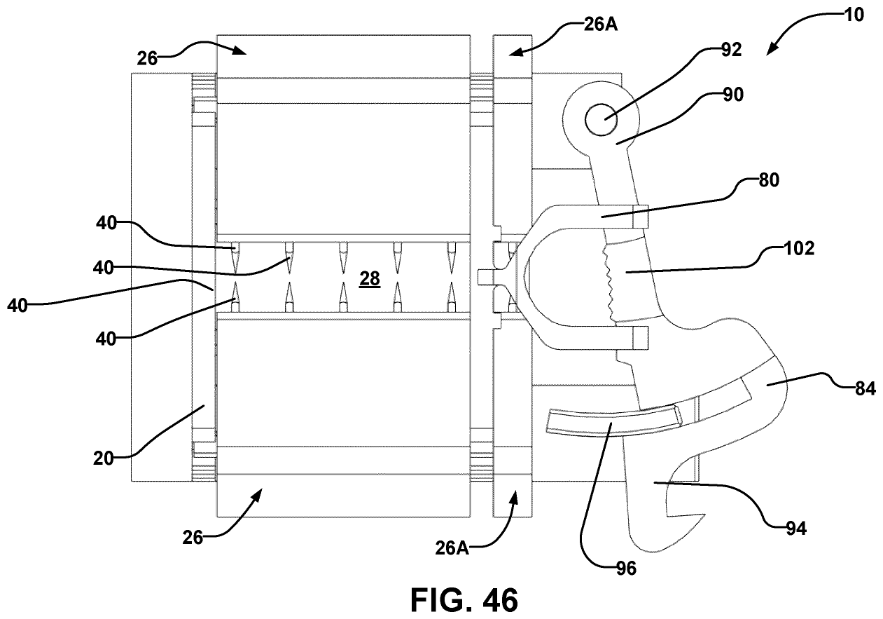

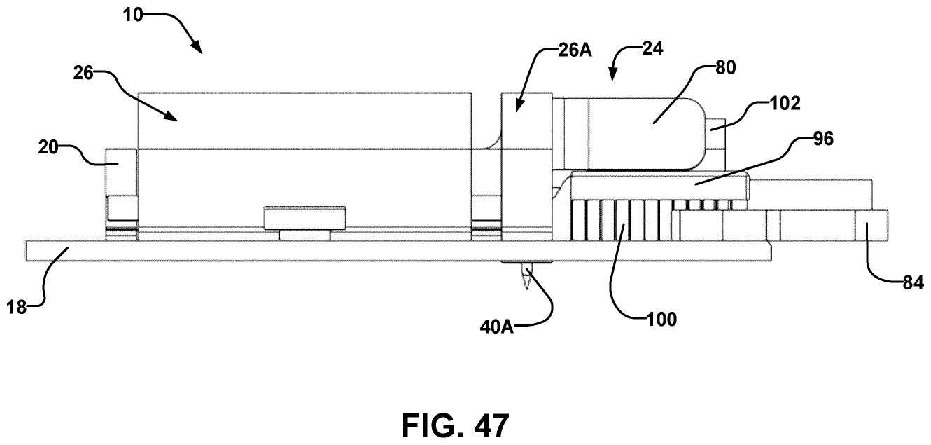

FIGS. 1-30 illustrate one exemplary embodiment of apparatus 10 and FIGS. 31-47 illustrate another exemplary embodiment of apparatus 10. With reference to FIGS. 1-3, in some embodiments apparatus 10 includes a frame 16 comprising a base 18 and a pair of spaced-apart sidewalls 20 projecting in an orientation generally perpendicular to base 18. For example, if base 18 is positioned in a generally horizontal orientation on a supine patient, sidewalls 20 project upwardly from base 18 in a generally vertical orientation. Apparatus 10 further includes a pivotable handle assembly 22 disposed between sidewalls 20 and a tube holder assembly 24 mounted adjacent one of the sidewalls 20 for securely and releasably holding tube 12. As shown best in FIGS. 1 and 27A-30B, apparatus 10 may be positioned with base 18 located on the skin surface of the patient adjacent incision 14 with sidewalls 20 oriented generally perpendicular to a longitudinal axis of incision 14. In some embodiments base 18 is generally U-shaped although many different shapes and configurations for supporting frame 16 on a patient are possible.

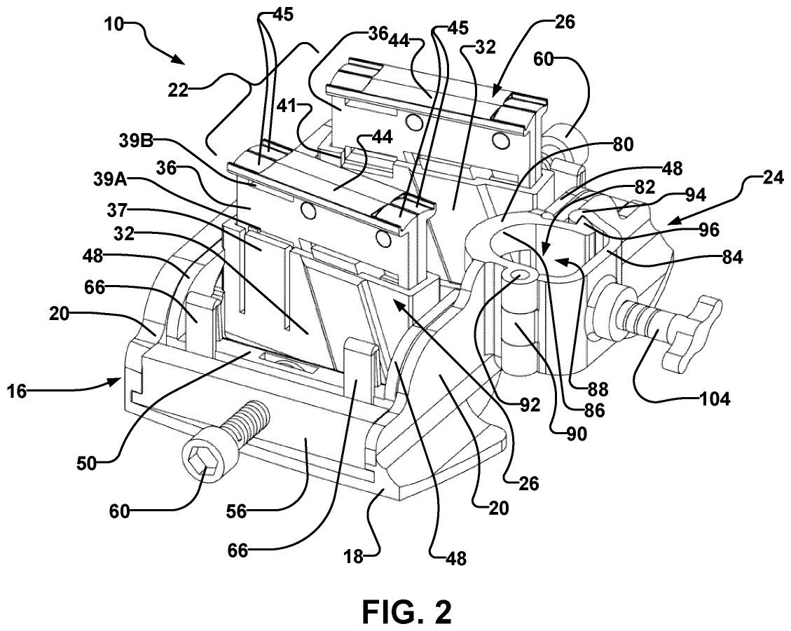

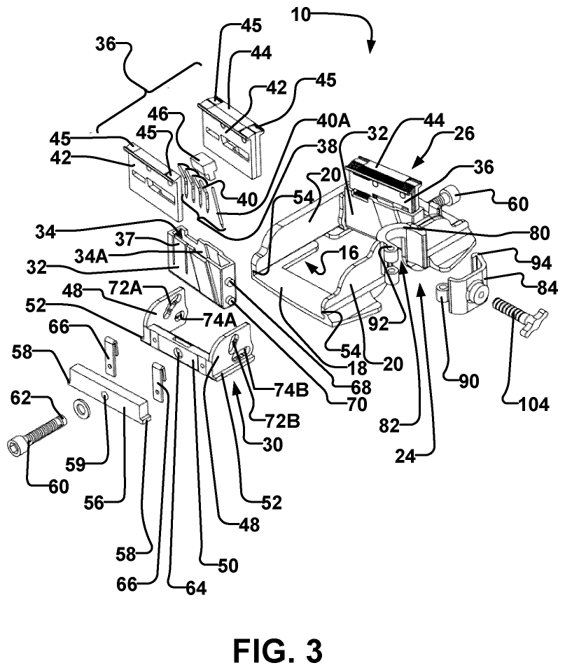

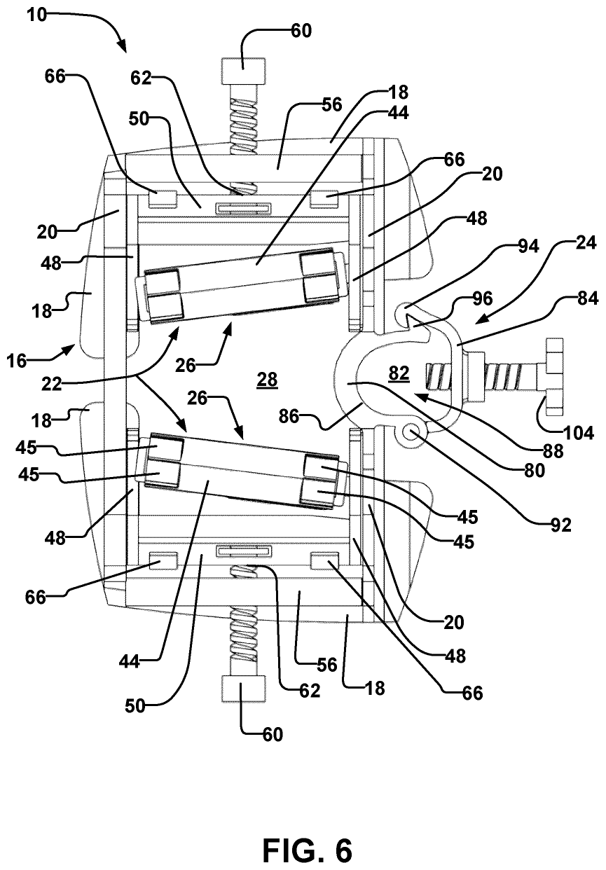

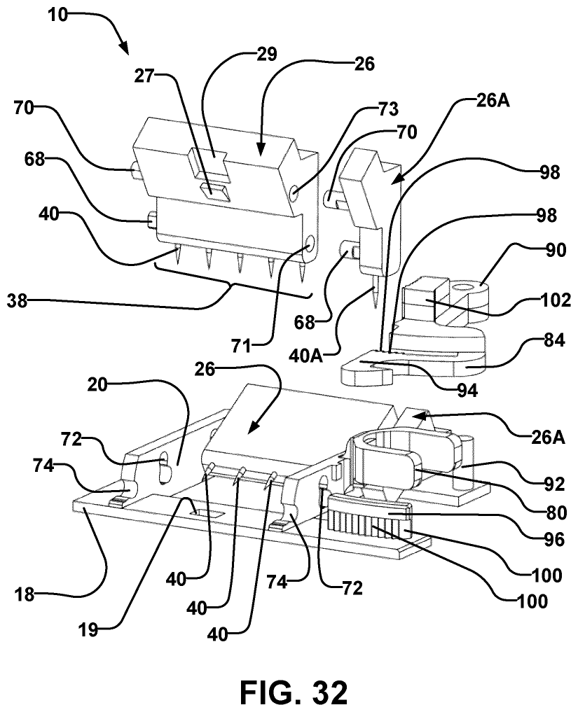

In some embodiments handle assembly 22 comprises a pair of spaced-apart handles 26 each configured for pivotable motion relative to frame 16. Handles 26 define a viewing space 28 therebetween (e.g. FIG. 6). In use, apparatus 10 may be oriented on the body of a patient with incision 14 located within viewing space 28 for easy visualization both during and after deployment of apparatus 10. In some embodiments handles 26 may each be coupled to a carriage 30 connectable to sidewalls 20 (e.g. FIGS. 2-3). In some particular embodiments handle carriages 30 may be slidably adjustable relative to sidewalls 20 to vary the relative spacing between handles 26. In some other embodiments handles 26 may be directly coupled to frame sidewalls 20 (e.g. FIG. 31-32).

In some embodiments each handle 26 comprises a housing 32 defining an inner compartment 34 open at either end and a needle cartridge 36 moveable within compartment 34. Cartridge 36 is configured to hold a needle array 38 comprising a plurality of spaced-apart needles 40. As discussed below, each needle 40 has a free end which projects from a lower surface of handle 26 in an engaged configuration for penetrating bodily tissue adjacent incision 14. Movement of each handle 26 to adjust the position of needles 40 and thereby deploy or remove apparatus 10 in the vicinity of incision 14 can be achieved by different means. In some embodiments each handle 26 is configured for manual manipulation by a user to adjust the position of the handle 26 between non-deployed/disengaged and deployed/engaged positions. For example, in some embodiments a user may manipulate each handle 26 to cause: (1) penetration of needles 40 into tissue of the patient adjacent to incision 14; (2) eversion (i.e. lifting) of the tissue engaged by the penetrated needles 40; and (3) closure of incision 14 by bringing together the everted tissue.

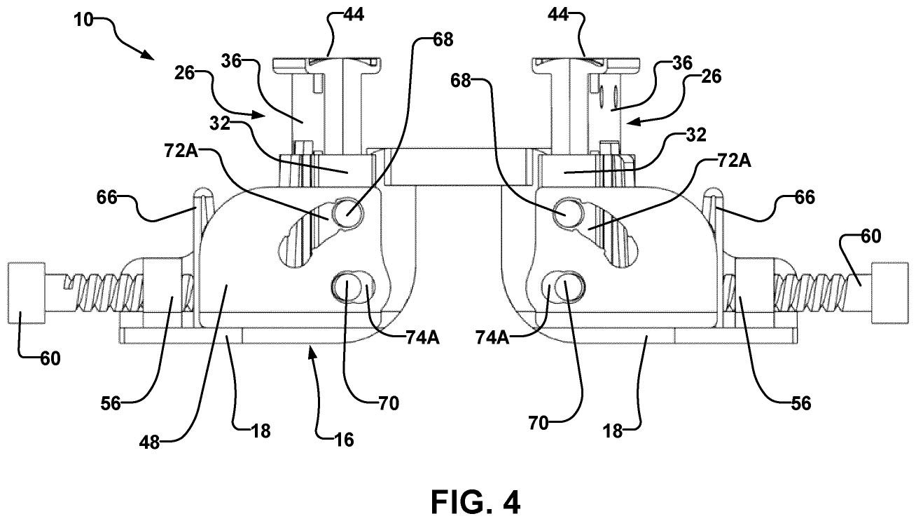

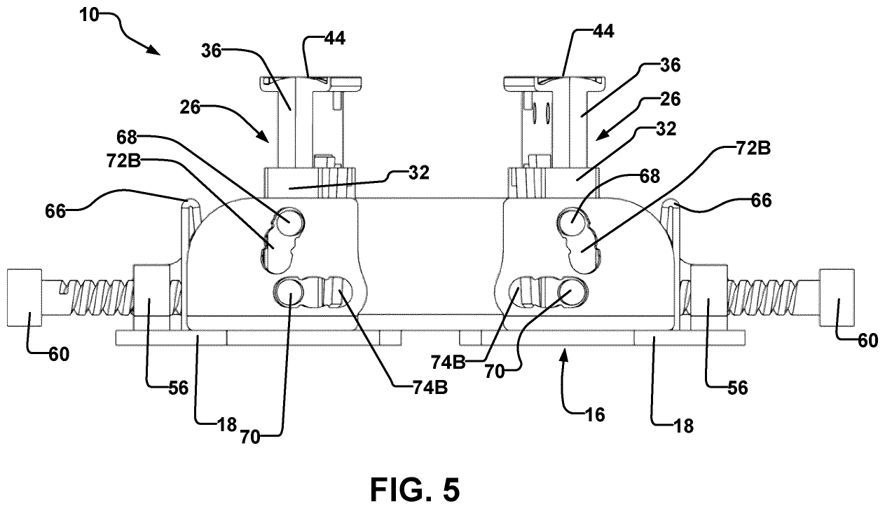

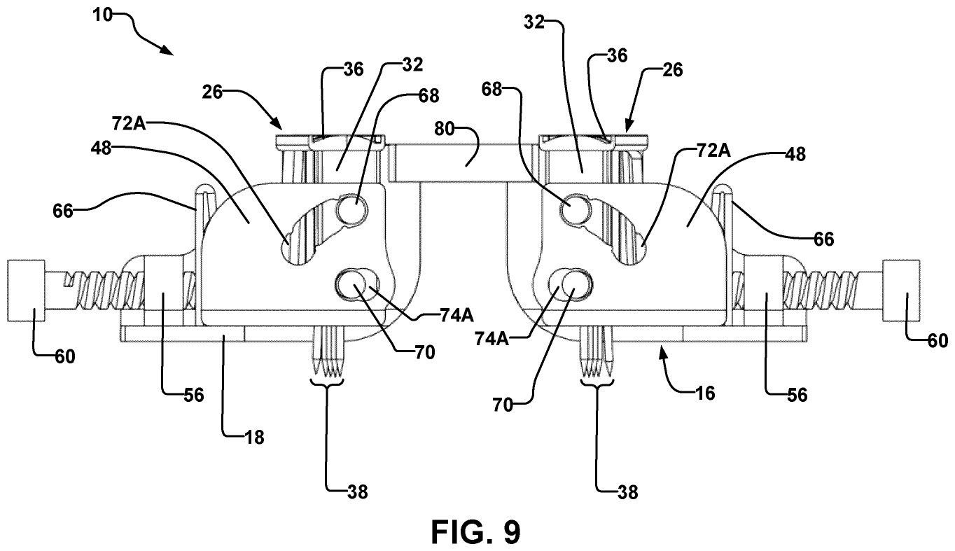

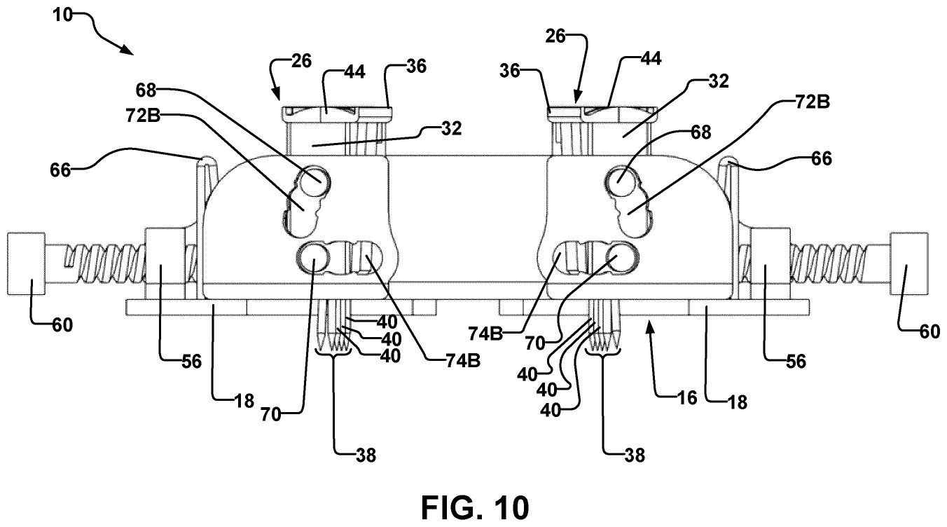

In some embodiments each needle cartridge 36 includes first and second sections 42 which may be coupled together for holding a needle array 38 therebetween (FIG. 3). For example, each needle 40 may be received within a slot or groove formed in an inner surface of one or more sections 42. When assembled, each cartridge 36 may comprise a flanged upper surface 44 having recessed end portions 45. Upper surface 44 can be depressed or raised by a user to effect movement of cartridge 36 within housing compartment 34. For example, in some embodiments each cartridge 36 is adjustable between: (1) an initial, disengaged position wherein needle array 38 is safely received within compartment 34 of a housing 32 to avoid inadvertent injury caused by needles 40; and (2) a, depressed, engaged position wherein needle array 38 projects from a lower surface of housing 32 for penetration into tissue of a patient adjacent incision 14. By way of illustration, FIGS. 4 and 5 show a pair of cartridges 36 in a disengaged position prior to deployment of apparatus 10 and FIGS. 9 and 10 show cartridges 36 in a depressed, engaged position during deployment of apparatus 10 (i.e. with the ends of needles 40 extending from apparatus 10 for penetrating bodily tissue).

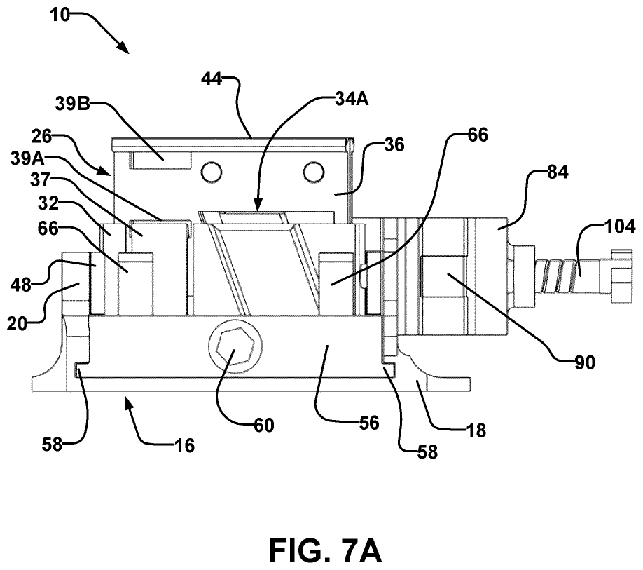

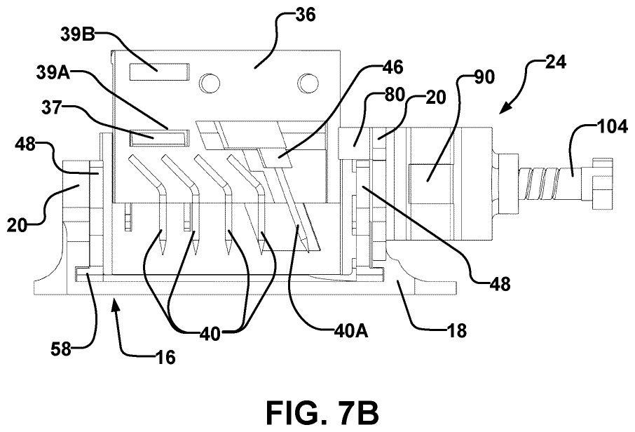

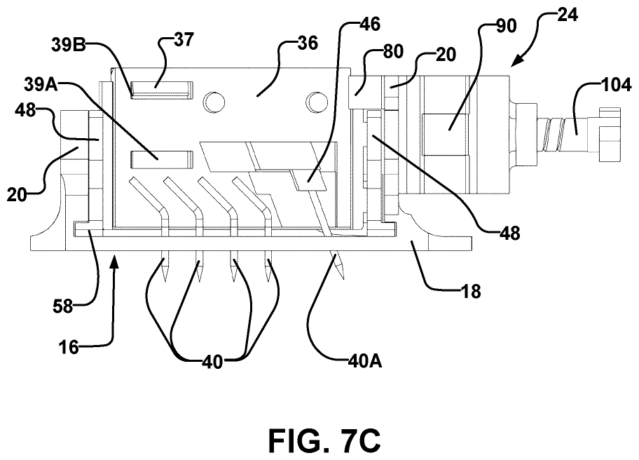

As indicated above, each cartridge 36 is movable relative to compartment 34 of a handle housing 32. In some embodiments housing 32 may include a tab 37 on an outer surface thereof which, in the disengaged position, is received in a slot 39A formed in cartridge 36 (FIGS. 2 and 7A-7B). When cartridge 36 is depressed within compartment 34 to the engaged position, tab 37 is released from slot 39A and travels along the surface of cartridge 36 until it is received in a slot 39B located adjacent upper surface 44, thereby releasably locking cartridge 36 in the engaged position (FIGS. 7C, 8 and 11). In the illustrated embodiment, cartridge 36 may be released from the locked position by inserting a thin tool through an access slot 41 formed on an opposite, inner surface of housing 32 to dislodge tab 37 from slot 39B. This enables readjustment of a cartridge 36 to the disengaged position within compartment 34, or complete removal of cartridge 36 from housing 32.

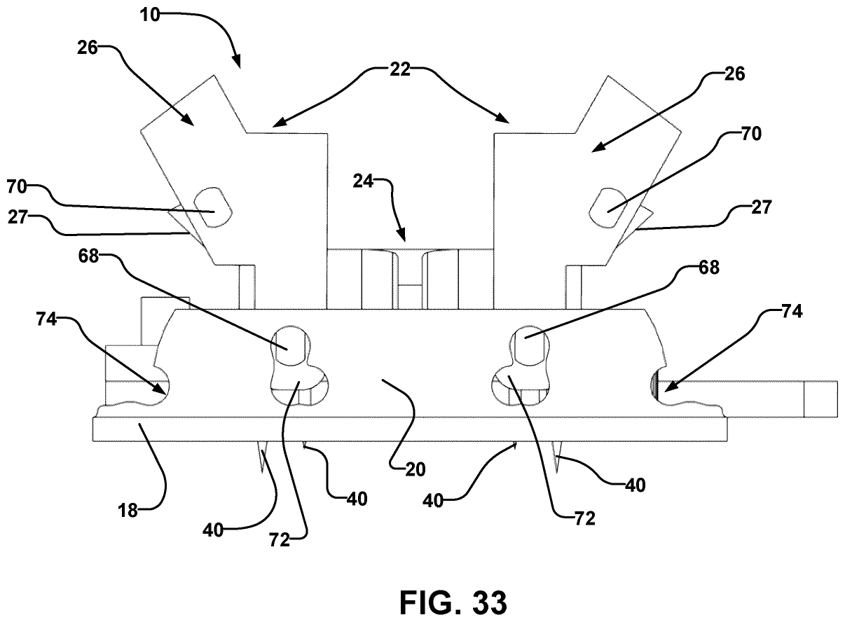

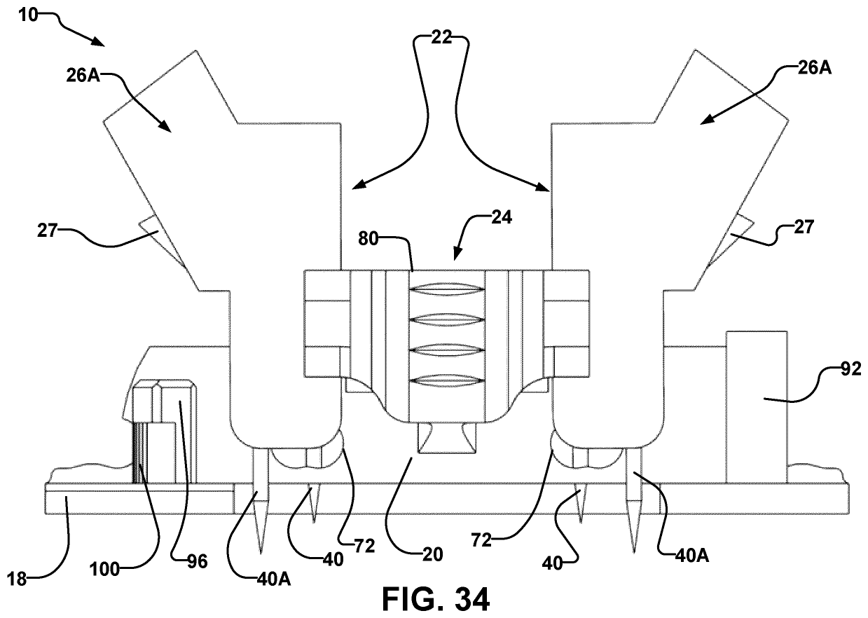

In some embodiments, a plurality of modular needle cartridges 36 may be provided, each removably insertable into a compartment 34 of a handle 26. This configuration enables different needle cartridges 36 to be selectively used on handles 26 depending upon the particular clinical application. For example, a needle cartridge 36 may be selected to suit different clinical conditions or factors, such as the age, sex, degree of obesity, degree of skin elasticity or other physical characteristics of a particular patient. For example, in the case of an infant or child, a cartridge 36 comprising a needle array 38 of relatively short needles 40 may be employed. In the case of an adult, a cartridge 36 comprising a needle array 38 of relatively long needles 40 may be employed. As will be apparent to a person skilled in the art, the number, spacing, length, curvature, orientation or other features of needles 40 may vary depending upon the intended use of cartridge 36. For example, needle array 38 may feature variable spacing between needles 40 or needles that are offset or non-aligned with the remainder of needles 40. In some embodiments cartridges 36 may be omitted and each needle array 38 may be directly mounted on a lower surface of a corresponding handle 26 (e.g. FIGS. 33-34). In different embodiments needle array 38 may be integrally or removably mounted on a respective handle 26.

In some embodiments the angle of insertion of one or more selected needles 40 within a needle array 38 may vary. For example, one or more needles 40A closest to the location of tube 12 (i.e. directly proximal to tube holder assembly 24) may be angled to better anchor needle array 38 in place. Positioning of angled needles 40A as close to tube 12 as possible helps to increase the surface contact between the patient's skin and tube 12, thereby improving the tissue seal at the tube insertion site. A better seal reduces the risk of air leakage into the pleural cavity and also improves fluid and air clearance. In some embodiments the angled needles 40A may be inserted at an angle within the range of about 20-40° relative to a vertical plane extending generally perpendicular to base 18 to a depth within the range of about 1-5 mm. In one particular embodiment the angle of insertion may be approximately 20-30°.

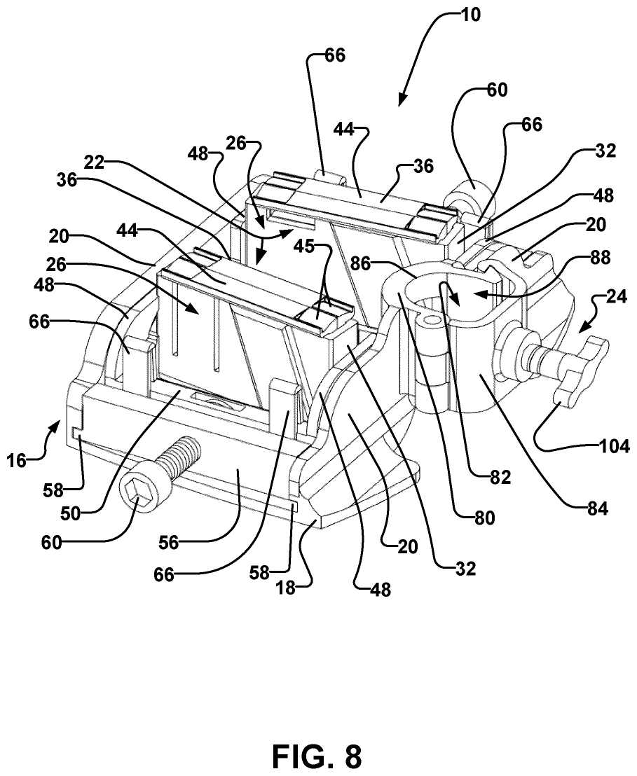

In the embodiment of FIGS. 1-30, each needle array 38 comprises a plurality of equal length, vertically oriented needles 40 and one angled needle 40A disposed at a location proximal to tube holder assembly 24. As indicated above, angled needle 40A is provided to ensure an effective tissue seal in the vicinity of tube 12. In some embodiments each angled needle 40A may be configured for movement with a needle guide 46. For example, guide 46 may be movable within an angled subcompartment 34A defined within housing compartment 34 (FIG. 3). As discussed below, downward movement of cartridge 36 within compartment 34 between needle disengaged (FIGS. 2 and 7B) and engaged (FIGS. 7C and 8) positions causes needle guide 46 to traverse within subcompartment 34A, causing needle 40A to penetrate the patient's tissue at a non-vertical angle. According to this exemplary mechanism, the angled needles 40A are advanced at the same time as the vertical needles 40 without the need for additional user input. FIGS. 7B and 7C are views of the interior of a needle cartridge 36 (with some parts of handle 26 removed for clarity) showing deployment of needle cartridge 36 and guide 46 from an initial, disengaged position with needles 40, 40A retracted within handle 26 (FIG. 7B) to a depressed, engaged position with needles 40, 40A extended from handle 26 (FIG. 7C). By way of example, in the embodiment of FIG. 7C angled needle 40A extends at an angle of approximately 20° relative to a vertical plane. As shown in FIGS. 7B-7C, as the user pushes down vertically to insert needle array 38, angled needle 40A translates horizontally from the disengaged position to the engaged position, enabling needle 40A to penetrate the skin proximate to tube 12 at the requisite angle.

After needles 40 have been deployed to engage bodily tissue adjacent incision 14 as described above, each handle 26 is adapted for movement relative to frame 16, either directly or indirectly. In some embodiments housing 32 of each handle 26 is pivotably coupled to a handle carriage 30. In some embodiments carriage 30 may include a pair of spaced-part carriage sidewalls 48 and an end wall 50 extending therebetween (FIG. 3). In some embodiments a pair of lower rails 52 project laterally from opposite sides of carriage 50. Rails 52 are received in channels 54 located in a lower portion of frame sidewalls 20. When carriage 30 is slidably inserted within frame 16, each carriage sidewall 48 is positioned immediately adjacent to a corresponding frame sidewall 20 (e.g. FIG. 2).

In some embodiments a non back-driveable variable adjustment mechanism may be provided for enabling adjustment of each handle carriage 30 relative to frame 16. For example, in some embodiments frame 16 may further include a pair of endwalls 56 extending between frame sidewalls 20 at opposite ends of apparatus 10. In some embodiments each endwall 56 includes lower lateral rails 58 configured to fit within channels 54. An adjustment screw 60 may be mounted on each endwall 56. Each screw 60 comprises an inner end 62 insertable through an aperture 59 formed in frame endwall 56 and connectable to a threaded aperture 64 formed in carriage endwall 50. As discussed further below, rotation of screw 60 actuates sliding movement of carriage 30 relative to frame 16 to vary the distance between handles 26. Screws 60 can thus be adjusted to cause a small amount of incremental movement of carriages 30 relative to fixed frame sidewalls 20, e.g. by turning each screw 60 to move carriages 30 the desired amount. This movement enables fine adjustment of the relative spacing of handles 26 (and hence the size of viewing space 28) either before or after handles 26 are closed and secured in an engaged position. For example, after handles 26 are closed and secured in place as discussed below, screws 60 may be adjusted to decrease the distance between handles 26 and thereby increase the pressure applied to the everted tissue at the site of incision 14, for example to ensure the integrity of the tissue seal and reduce the risk of infection Conversely, screws 60 may alternatively be adjusted to increase the distance between handles 26 and thereby reduce the pressure applied to the everted tissue at the site of incision 14. For example, in some cases it may be desirable to reduce the tissue pressure at the site of incision 14 to avoid ischaemic conditions and reduce the risk of tissue necrosis. Such post-closure fine adjustments are particularly advantageous if apparatus 10 is deployed on a patient for an extended period of time and/or if the patient is frequently moving during the treatment period. In some embodiments handle carriages 30 could also be configured for post-closure vertical adjustment in addition to horizontal adjustment.

Movement of handle assembly 22 relative to frame 16 to adjust the position of needles 40 and thereby deploy or remove apparatus 10 at the vicinity of incision 14 can be achieved by various different means. In some embodiments pivoting motion of handle assembly 22 relative to frame 16 is constrained by a pair of pivot pins 68 and 70 projecting laterally from each handle 26. More particularly, in some embodiments pins 68 and 70 project laterally from handle housing 32 and comprise end portions which are received in corresponding guide slots 72 and 74 formed in a sidewall 48 of carriage 30. In the side views of apparatus 10 (e.g. FIGS. 4 and 5) portions of frame 16 and tube holder assembly 24 have been omitted to more clearly show movement of pins 68, 70 within corresponding slots 72, 74. As discussed further below, slots 72 and 74 may optionally be formed in frame sidewalls 20 if movable carriages 30 are omitted. Each slot 72, 74 is sized and shaped to guide the range of permitted travel of pivot pins 68, 70 and hence the range of permitted motion of handles 26. For example, slots 72, 74 may be sized and shaped to permit at least two degrees of movement of needles 40, e.g. downward vertical movement to penetrate the user's tissue and pivotable movement to evert (lift) the penetrated tissue and bring the everted tissue together to close incision 14.

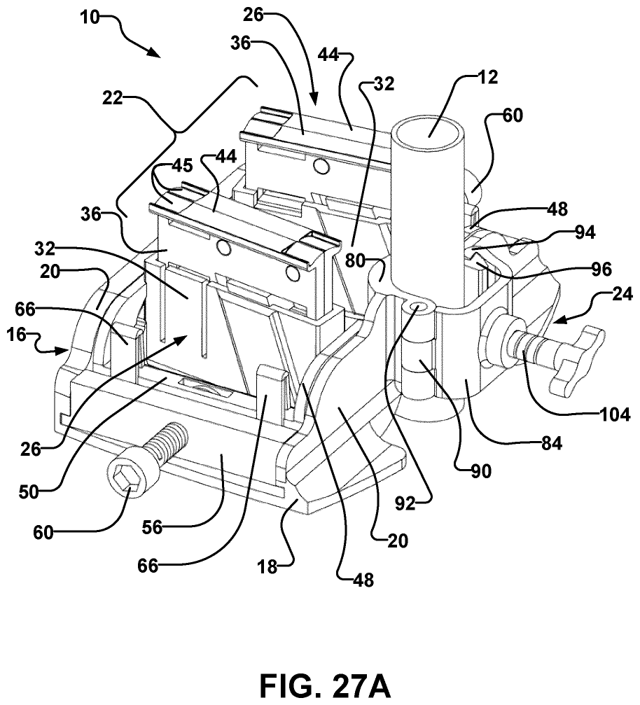

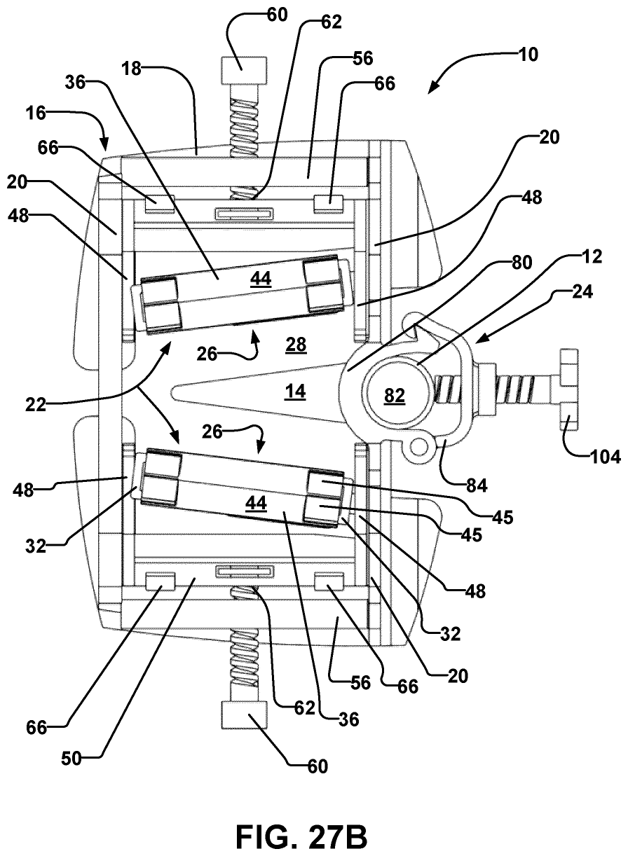

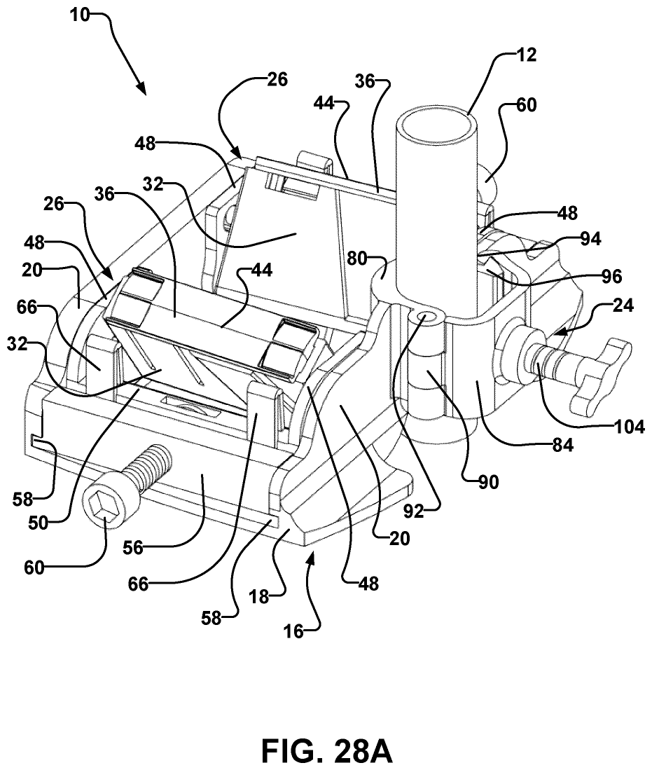

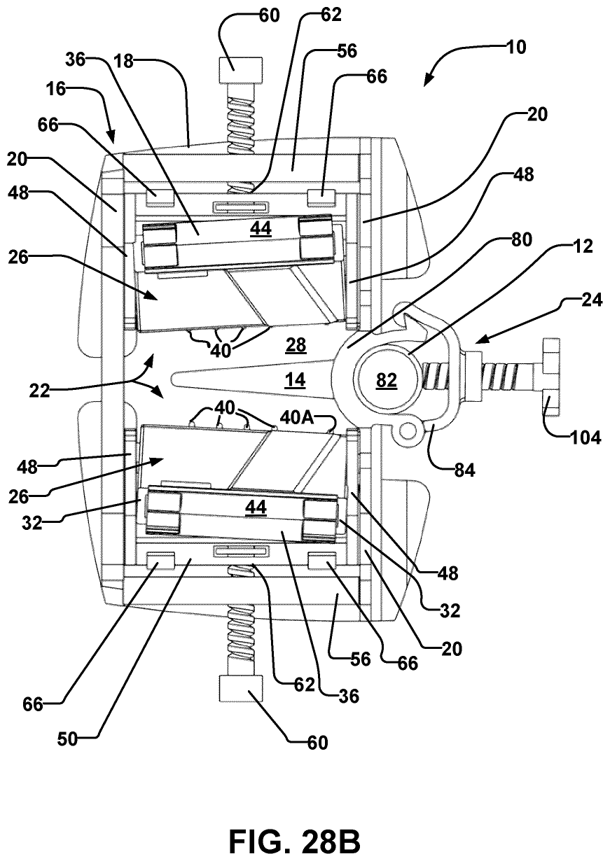

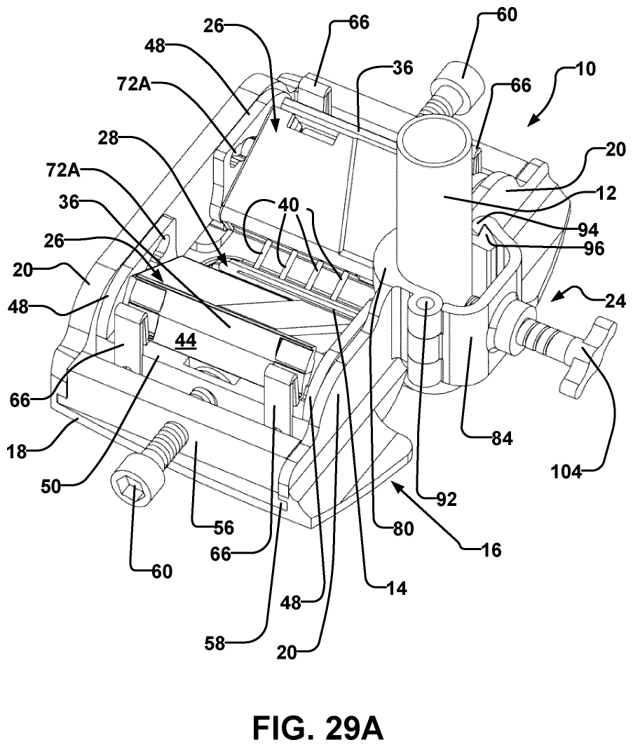

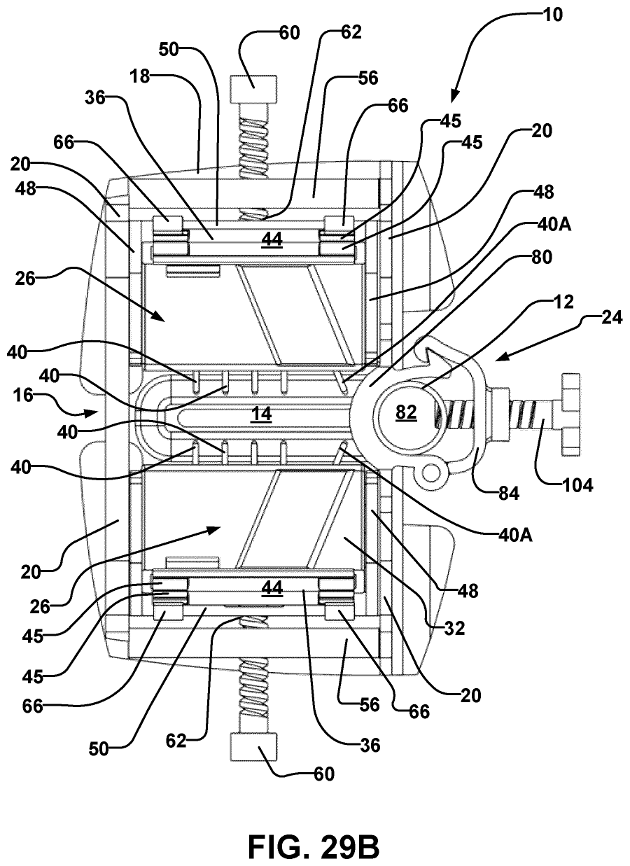

In some embodiments slots 72, 74 may be configured so that the amount of travel of handles 26 can vary over the length thereof depending upon relative proximity to tube holder assembly 24. As best shown in top plan view (e.g. FIG. 27B), in an initial non-deployed position the relative distance between handles 26 varies between a maximum distance proximal tube holder assembly 24 to a minimum distance distal from tube holder assembly 24. Thus, in plan view handles 26 extend in non-parallel planes and the viewing space 28 between handles 26 is generally wedge-shaped rather than rectangular-shaped. As will be appreciated by a person skilled in the art, this configuration is desirable since the width of the opening of incision 14 varies along the length thereof, i.e. the width of incision 14 must be larger adjacent tube holder assembly 24 to accommodate the diameter of tube 12 which passes therethrough (FIG. 27B). When handles 26 are inserted into the patient's tissue, pivoted to cause eversion of the tissue and moved together to secure handles 26 in a closed position (e.g. FIGS. 28A-29B), the portion of each handle 26 proximal to tube holder assembly 24 travels a greater distance than the portion of each handle 26 distal from tube holder assembly 24. This ensures incision 14 is effectively sealed along its entire length, including in the region immediately adjacent to tube 12. Thus in a fully deployed configuration handles 26 are aligned in generally parallel planes, the spacing between handles 26 is relatively constant along the length thereof, and space 28 enabling visualization of the closed incision 14 is generally rectangular-shaped in plan view (FIG. 29B).

In some particular embodiments the size, shape and orientation of guide slots 72, 74 may vary depending on whether they are located distal or proximal to tube holder assembly 24 and hence tube 12. In the drawings relating to the embodiment of FIG. 1-30, the distal slots are identified as 72A and 74A (e.g. FIGS. 3 and 4) and the proximal slots are identified as 72B and 74B (e.g. FIGS. 3 and 5). As indicated above, since the width of incision 14 is ordinarily larger proximal to tube 12 to accommodate the width thereof (FIG. 27B), proximal slots 72B, 74B are sized and shaped to permit a larger degree of travel of handles 26 proximal to tube 12.

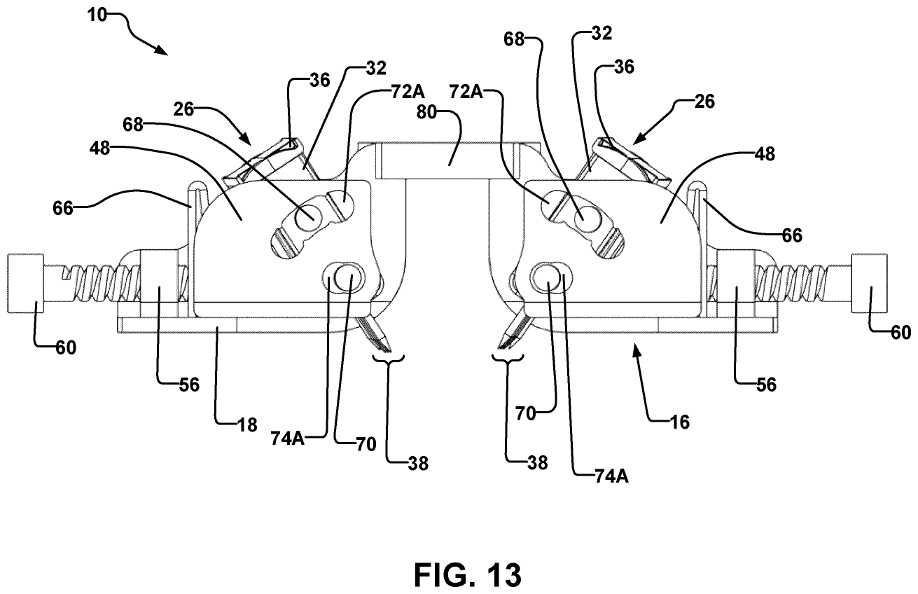

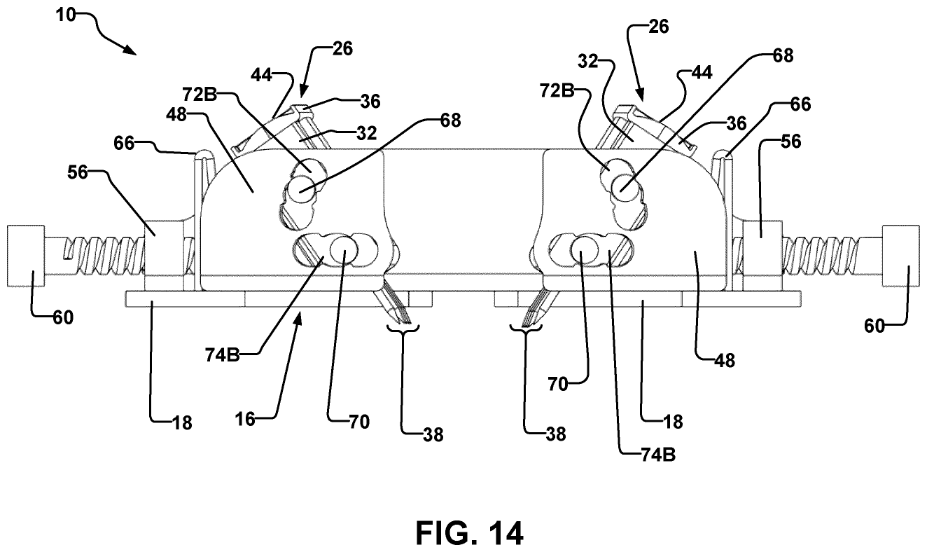

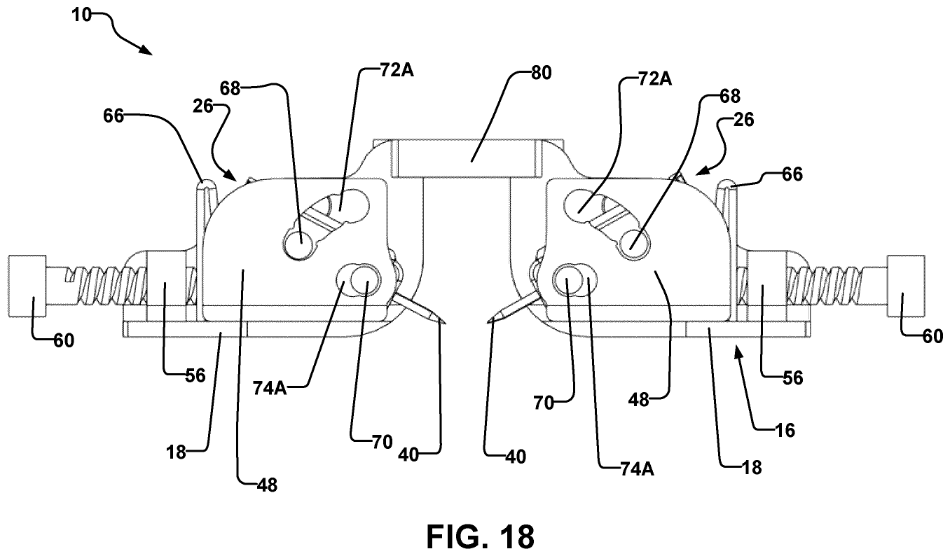

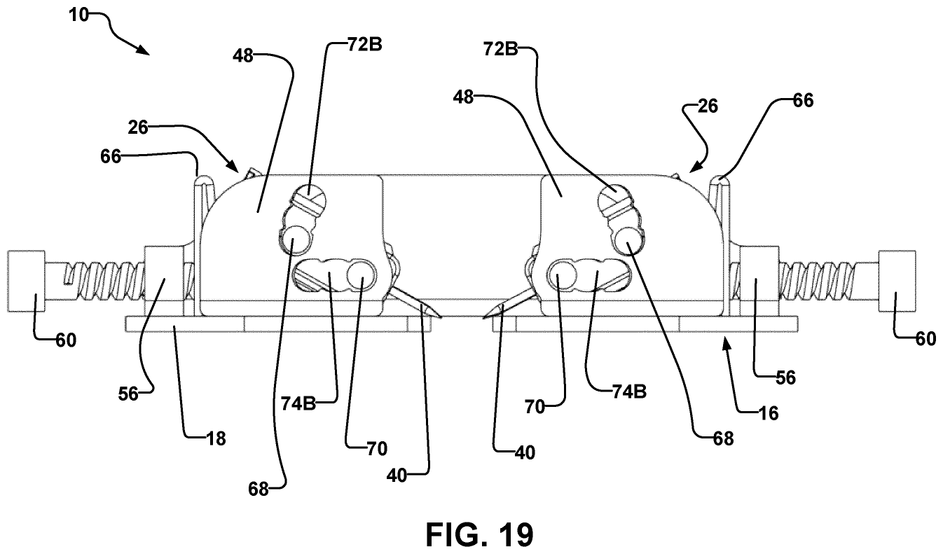

Initially, in this example embodiment, when handles 26 are in a non-deployed configuration, pivot pins 68, 70 are in the positions shown in FIGS. 4 and 5. That is, pivot pins 68 are located in respective upper portions of slots 72A and 72B and pivot pins 70 are located in respective outer portions of slots 74A and 74B. After cartridges 36 are depressed within respective housing compartments 34 to adjust each needle array 38 from a disengaged position to an engaged (FIGS. 9 and 10), handles 26 are pivoted to a partially deployed position to partially close incision 14 (FIGS. 13 and 14). In the partially deployed position pivot pins 68 are located in respective mid-portions of slots 72A and 72B and pivot pins 70 are in respective mid-portions of slots 74A and 74B. It is apparent by comparing FIGS. 13 and 14 that pivot pins 70 travel a greater distance inwardly, i.e. toward incision 14, within proximal slot 74B than distal slot 74A, thereby effecting variable inward travel of handles 26 along the length thereof, as discussed above. Further pivoting motion of handles 26 relative to frame 16 to a fully deployed position (FIGS. 18 and 19) causes further eversion of the tissue engaged by needles 40 and substantially complete closure of incision 14. In the fully deployed position pivot pins 68 are located in respective lower portions of slots 72A and 72B and pivot pins 70 are in respective inner portions of slots 74A and 74B. In some embodiments it may be necessary to press handles 26 slightly inwardly toward one another in the fully deployed configuration to fully seat pivot pins 70 within the innermost portion of slots 74A and 74B, effectively sealing incision 14 and securing handles 26 in place. A pair of spaced-apart locking clips 66 may be disposed between endwalls 50, 56 for releasably engaging a handle 26 in a fully deployed position.

In particular, in some embodiments a free end of each clip 66 is received in a recessed end portion 45 of upper surface 44 of cartridge 36 in a fully deployed position (e.g. FIG. 29B), thereby securely locking handle 26 to frame 16. As discussed above, depending upon the desired pressure to be applied to the everted tissue in contact at the site of incision 14, the relative position of handles 26 may be incrementally adjusted in the fully deployed position by turning screws 60, causing moveable carriages 30 to advance (i.e. either inwardly or outwardly) relative to fixed frame 16 the desired distance.

As will be appreciated by a person skilled in the art, many other means for guiding and controlling the movement of handle assembly 22 relative to frame 16, and hence the travel of needles 40, between disengaged/non-deployed and engaged/deployed positions, may be envisioned.

As discussed above, tube holder assembly 24 is provided to securely and releasably hold tube 12, for example at one end of incision 14. As shown in FIGS. 2 and 3, in some embodiments tube holder assembly 24 comprises a tube guide 80 defining a space 82 for receiving tube 12 and a locking arm 84 for releasably holding tube 12 within space 82. In some embodiments tube guide 80 is generally C-shaped, having a curved closed end 86 mounted on a frame sidewall 20 and an open end 88 to enable tube 12 to be placed in or removed from guide 80 when tube holder assembly 24 is in an open position.

In some embodiments locking arm 84 may be curved or otherwise contoured to mate with the contour of guide 80 (FIG. 3) and capture tube 12 therebetween. In some embodiments a first end 90 of locking arm 84 is pivotably coupled to a post 92 to enable adjustment of arm 84 between open and closed positions. In the closed position, a second end 94 of arm 84 may engage a rib 96 located at an end portion of tube guide 80 or projecting from a frame sidewall 20. In some embodiments guide 80 is centered on a transverse centreline of apparatus 10 generally in alignment with the longitudinal axis of incision 14 (FIG. 29B).

In some embodiments an adjustment mechanism, such as a threaded screw 104, may be employed to adjust the degree of compressive force applied to tube 12 captured within space 82. Such a screw 104 may be configured for fine adjustments or a combination of coarse and fine adjustments. Depending upon the precise position and configuration of locking arm 84 or screw 104, the compressive force applied to tube 12 may vary. In some embodiments, guide 80 is designed to accommodate a range of tube sizes from 8 Fr to 24 Fr. A gauge may be employed to indicate the appropriate clamping force depending upon the size of tube 12 in question.

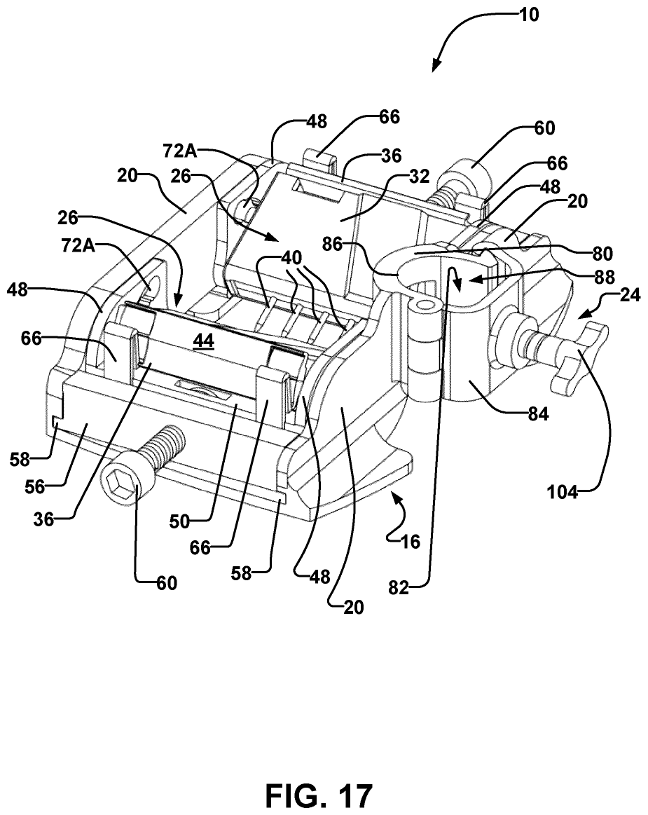

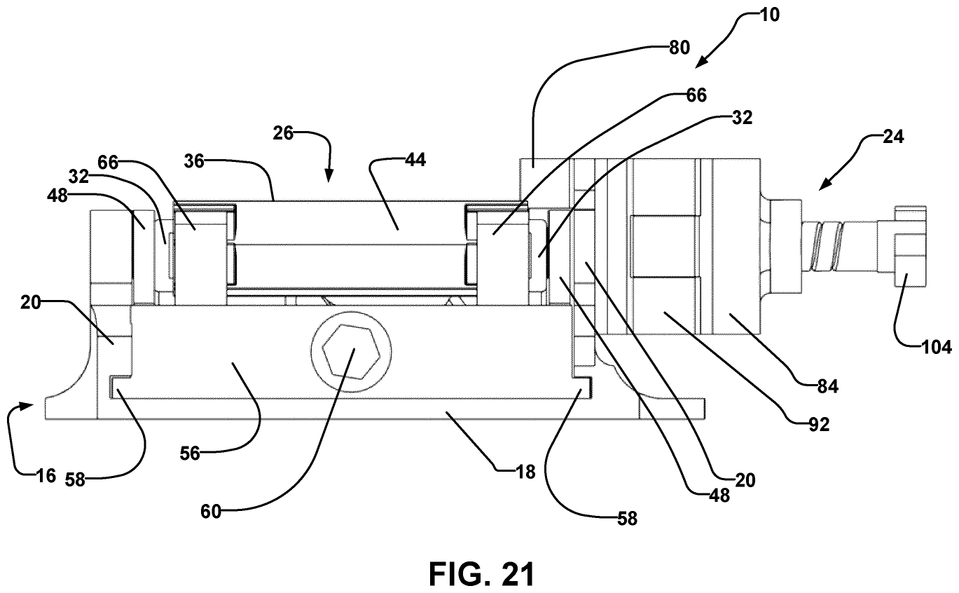

As shown, for example, in FIGS. 17 and 21, apparatus 10 has a relatively low profile in its fully deployed configuration to ensure that it is less obtrusive, lighter and more comfortable for deployment for extended periods of time than some prior art tube holder devices, which may particularly be of importance in the case of patients frequently moving between supine, sitting and standing positions. For example, the height of apparatus 10 in the deployed configuration may be less than approximately 5 cm or in some embodiments less than 3 cm. Tube holder assembly 24 is engineered to securely hold tube 12 in place for as long as is required, which is anticipated to be up to approximately 30 days. If necessary, locking arm 84 can be released and tube 12 can be relocated or repositioned as required during the treatment period. After the tube position adjustment, locking arm 84 can then be reset to fix tube 12 in place.

In operation, apparatus 10 is configured to securely and releasably hold a tube 12 inserted through an incision 14 or other wound or temporary body opening of a patient undergoing treatment. As shown, for example, in FIGS. 1 and 27A-30B, incision 14 may be an elongated incision formed in the chest wall of the patient by a surgeon. Incision 14 may alternatively be a puncture wound or other wound caused by a bodily injury. Apparatus 10 enables a physician or other trained user to: (1) securely hold tube 12 at the desired location and orientation extending through incision 14; and (2) close incision 14 without the use of sutures, adhesives and/or other medical instruments. Apparatus 10 is intended to reduce the time and skill required to reliably close incision 14 after optimum placement of tube 12 without necessarily requiring the participation of an attending physician.

FIGS. 27A-30B illustrate an exemplary procedure for deploying apparatus 10 on a patient. After formation of incision 14 with a scalpel or other instrument at the desired body location, a tube 12 of the desired length and diameter is passed through incision 14 into the interior of the patient's body. Ordinarily the first step in the deployment of apparatus 10 is for the physician or other trained user to hold tube 12 in place (e.g. with a single hand) ensuring that it does not move in or out of incision 14. Tube 12 is then moved laterally to one end of incision 14. The physician or other trained user then positions apparatus 10 (e.g. using a free hand) such that base 18 of frame 16 is located on the surface of the patient's skin adjacent incision 14 and tube 12 is received within guide 80 of tube holder assembly 24. Optionally, tube 12 may then be secured within guide 80 by closing locking arm 84 and adjusting screw 104. As shown for example in FIGS. 27A and 27B, in some embodiments frame sidewalls 20 extend generally perpendicular to incision 14 with pivotable handle assembly 22 extending therebetween. Apparatus 10 is oriented so that viewing space 28 defined between handles 26 is aligned with incision 14 to enable visualization of incision 14 throughout the entire deployment procedure (and during subsequent wear by the patient).

Handles 26 are initially disposed in a non-deployed, disengaged position with needle array 38 comprising spaced-apart needles 40 removed from the patient's skin in some embodiments. Apparatus 10 is held firmly in place and handles 26 are then engaged to insert needles 40 of needle array 38 into the patient's tissue adjacent incision 14. In the embodiment of FIGS. 1-30, this step can be accomplished by manually depressing upper flange 44 of cartridge 36 toward the patient, e.g. downwardly if the patient is in a supine position, to fully insert each cartridge 36 within a corresponding compartment 34 of housing 32 (e.g. FIGS. 9 and 10). This causes needles 40 to extend below base 18 of frame 16 to penetrate generally vertically into the patient's tissue adjacent incision 14. In some embodiments needles 40 are inserted into the tissue to a depth of between 1 mm and 10 mm.

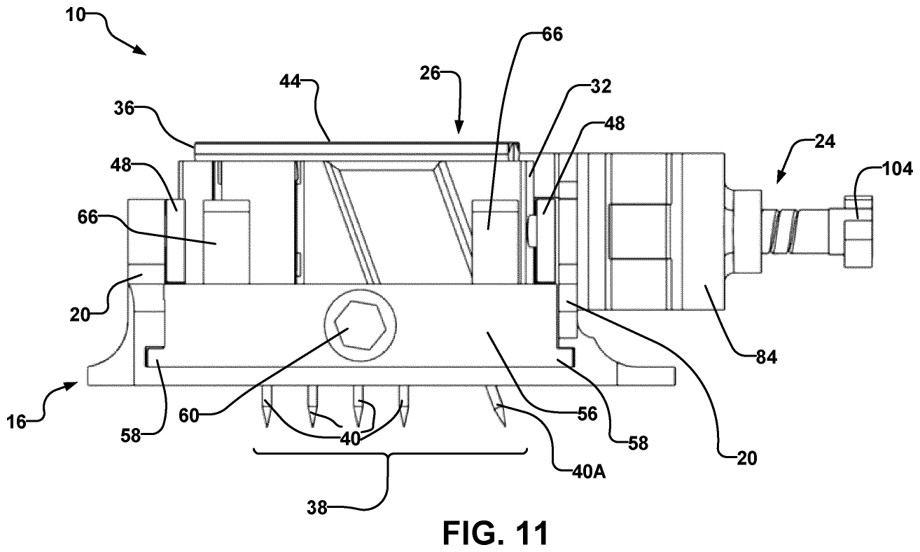

As discussed above, one or more needles 40A closest to the location of tube 12 (i.e. directly proximal to tube holder assembly 24) may be angled to better anchor needle array 38 in place (FIG. 11). Positioning of angled needles 40A as close to tube 12 as possible helps to increase the surface contact between the patient's skin and tube 12. In some embodiments angled needles 40A may be configured for movement within a needle guide 46 and may be inserted into the patient's tissue at an angle within the range of about 20-40°.

After needles 40 are fully inserted, handles 26 are then pivoted to cause eversion (lifting) of the patient's tissue on either side of incision 14 to provide optimal conditions for tissue closure. As described above, pivoting motion of handles 26 is constrained by the extent of travel of pivot pins 68, 70 within guide slots 72, 74 (72A, 72B; 74A, 74B) formed in carriage sidewalls 48 (e.g. FIGS. 9 and 10) and/or sidewalls 20. In some embodiments handles 26 may pivot within a range of approximately 30-90°. As discussed above, in some embodiments slots 72, 74 may be configured so that the amount of travel of handles 26 varies over the length thereof depending upon relative proximity to tube holder assembly 24. This feature is desirable since the width of incision 14 ordinarily varies along the length thereof, i.e. the width of incision 14 is larger adjacent to tube holder assembly 24 to accommodate the diameter of tube 12 passing therethrough. Thus in some embodiments the amount of travel of handles 26 increases with proximity to tube 12. As shown best in top plan view (FIGS. 27B, 28B and 29B), in some embodiments this variable amount of travel of handles 26 during the pivoting/eversion step causes the handles 26 to move from an initial pivot position extending in non-parallel planes to a final pivot position extending in generally parallel planes. More particularly, handles 26 may be pivoted from the generally vertical initial position of FIG. 27A to the partially deployed position of FIG. 28A to the fully deployed position of FIG. 29A. This pivotable motion causes incision 14 to close along the entire length thereof as respective needle arrays 38 are brought together. In the illustrated embodiment, handles 26 move from an initial position extending in non-parallel planes (FIG. 27B) to the fully deployed position extending in generally parallel planes (FIG. 29B).

After the patient's tissue is fully everted and incision 14 is substantially closed as described above, in some embodiments handles 26 are moved toward one another in a further closure step to secure apparatus 10 in a closed position with the everted tissue in contact, thereby sealing incision 14. In some embodiments pivot pins 68, 70 are releasably locked in a corresponding slot or slot portion in the closed position. In this closure step, handles 26 may be moved toward one another in a common (e.g. horizontal) plane a distance of between about 1 mm to 10 mm. In some embodiments a free end of each clip 66 is received in a recessed portion 45 of upper surface 44 of cartridge 36 in a fully deployed position to securely lock each handle 26 to frame 16 (e.g. FIG. 29B). Needles 40, which are intended to remain embedded in the patient's tissue during the full duration of deployment of apparatus 10, create a strong mechanical connection or “anchor” between apparatus 10 and the patient's tissue. Although apparatus 10 is securely locked in the closed position, the locking mechanism can be reset if and when required by reversing the deployment steps.

As described above, before incision 14 is closed and sealed by deploying apparatus 10, tube 12 may be fixed in place within guide 80 by closing locking arm 84. Optionally, in some embodiments tube 12 may be fixed in place within guide 80 after incision 14 is closed and sealed or after it is partially closed. If necessary, locking arm 84 can be released and tube 12 can be relocated or repositioned as required during the treatment period. Thus tube holder assembly 24 is readably adjustable. After any tube position adjustment, locking arm 84 can then be reset to once again fix tube 12 in place as described above.

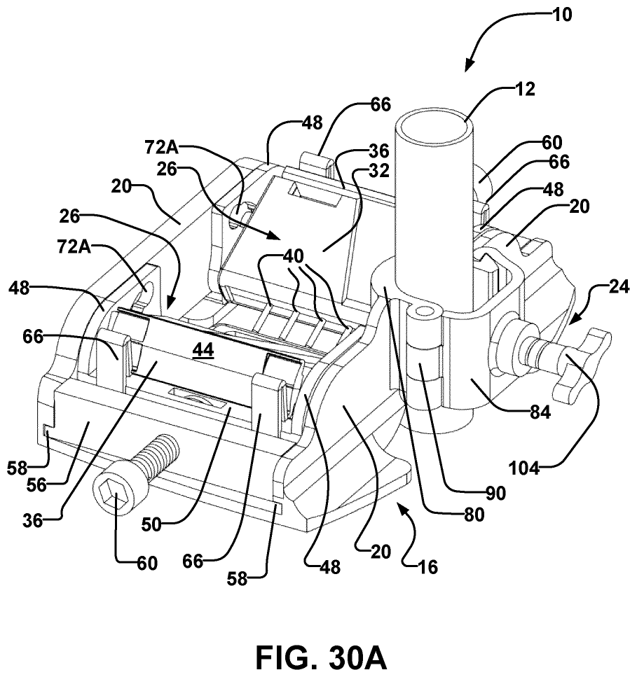

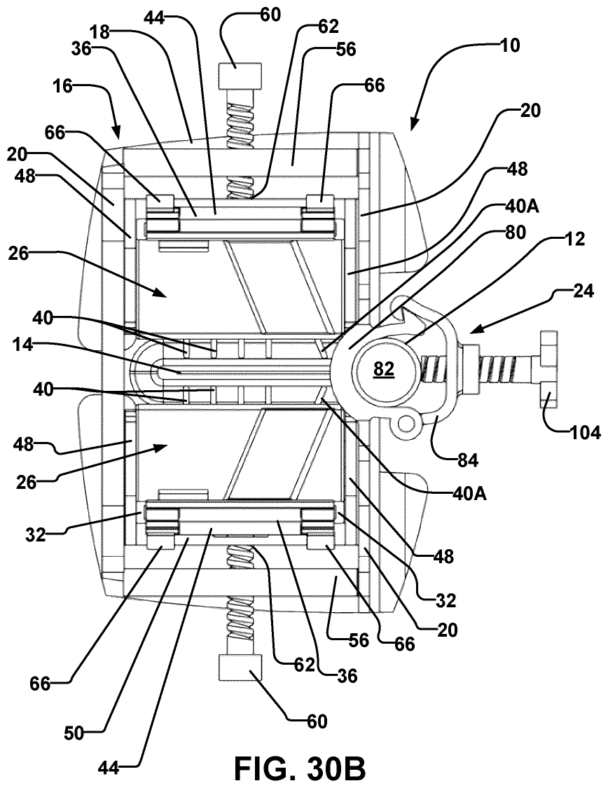

In some embodiments apparatus 10 may also include an incision closure adjustment mechanism to enable post-closure fine adjustment of the relative spacing between handles 26 and hence the distance between needle arrays 38 (FIGS. 30A and 30B). As described above, in some embodiments a non back-driveable variable adjustment mechanism, such as screws 60, may be adjusted to actuate movement of handle carriages 30 relative to fixed frame sidewalls 20. For example, small incremental adjustment of handle carriages 30 in a generally horizontal plane may be desirable to increase the pressure applied to the everted tissue at the site of incision 14 to ensure the integrity of the tissue seal. Conversely, in some cases it may be desirable to reduce the tissue pressure at the site of incision 14 to avoid ischaemic conditions and reduce the risk of tissue necrosis. Such post-closure fine adjustments are particularly advantageous if apparatus 10 is deployed on a patient for an extended period of time and/or if the patient is frequently moving during the treatment period. In some embodiments locking arm 84 and/or screw 104 may be similarly adjustable to alter the forces applied to tube 12 held within tube guide 80. Thus after apparatus 10 is deployed as described above, both forces applied to everted tissue at the site of incision 14 and forces applied to tube 12 can be incrementally and independently adjusted to an optimum degree.

As discussed above, an important feature of apparatus 10 is that it enables visualization of incision 14 both during the deployment and recovery processes. Incision 14 is visible throughout within viewing space 28 defined between handles 26. Optimally, it is desirable for care providers to be able to observe and assess the status of incision 14 post-deployment of apparatus 10 in order to monitor the rate of healing and to avoid infection. In some embodiments apparatus 10 could be integrated with specific wound dressings to provide optimum functionality and efficiency for the care provider.

Apparatus 10 may be easily removed from a patient after completion of treatment by opening locking arm 84 and removing tube 12 from guide 80. Needles 40 can be removed from the patient's tissue by reversing the closure, eversion and insertion steps described above.

In some embodiments apparatus 10 can be configured for use on different patients having different physical or clinical characteristics. For example, different modular needle cartridges 36 having different needle arrays 38 may be used on handles 26 to suit particular applications. For example, the length, spacing, angle, degree of curvature or other characteristics of needles 40 could vary as between different cartridges 36. Thus apparatus 10 may be easily customized to suit, for example, neonatal, geriatric, obese or other patient types.

In another exemplary embodiment of the invention illustrated in FIGS. 31-47, adjustable handle carriages 30 and needle cartridges 36 have been omitted. In this embodiment needle array 38 is mounted directly on a lower portion of handles 26, and optionally a supplementary handle portion 26A, and slots 72 for receiving end portions of pivot pins 68 are formed directly in frame sidewalls 20. In the illustrated embodiment, slot 72 is generally L-shaped. In a fully deployed configuration, pin 70 is received in a slot 74 formed in an end portion of sidewall 20 rather than in an intermediate portion thereof. In the illustrated embodiment slots 72, 74 are the same size and shape on sidewalls 20 proximal and distal to tube holder assembly 24 (FIG. 32) and hence respective side portions of handles 26 will pivot in unison and travel over the same distance to bring the everted tissue together. However, in other embodiments slots 72, 74 may be sized and shaped as described above to effect variable travel of handles 26 over the length thereof.

As shown in FIG. 32, each handle 26 may include a wedge-shaped locking tab 27 which is configured to be received in a slot 19 formed in base 18 when handle 26 is pivoted to a fully closed position. In the illustrated embodiment a cut-away opening 29 is formed in handle 26 to permit access to locking tab 27 in the closed position.

In the embodiment of FIGS. 31-47, the needle 40A closest to tube holder assembly 24 is longer in length than the other needles 40 and is mounted on a supplementary handle portion 26A. In this embodiment, needle 40A is longer proximal to tube assembly holder 24 to better engage and seal tissue at the end of incision 14 receiving tube 12. In this embodiment a pivot pin 68 proximal to tube holder assembly 24 is mounted on supplementary handle portion 26A and extends through slot 72 and is received in an aperture 71 formed in handle 26. In an initial position, pivot pin 70 extends above handle sidewall 20 and is received in an aperture 73 formed in handle 26 on one side thereof (FIG. 32).

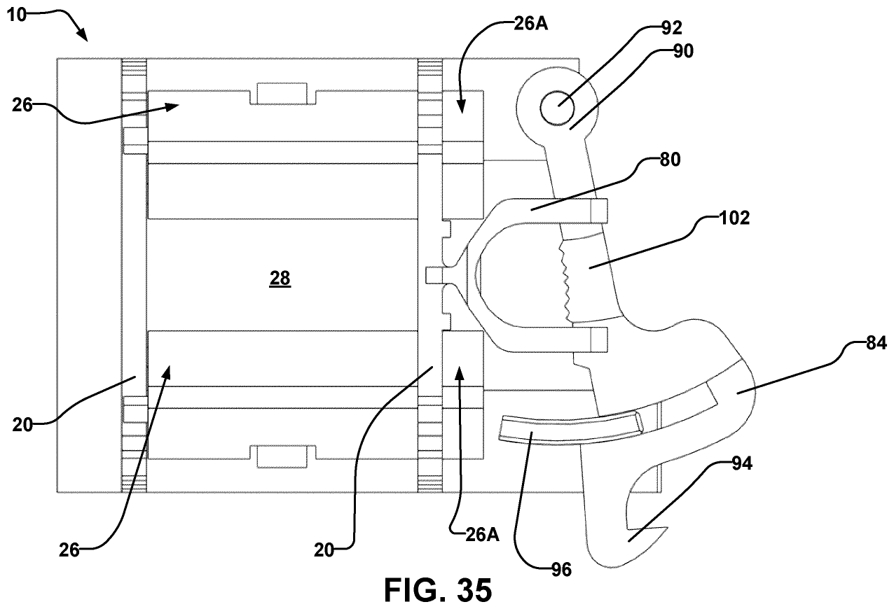

In the embodiment of FIGS. 31-47, tube holder assembly 24 is configured and functions in a manner generally similar to the embodiment of FIGS. 1-30 and also comprises an adjustment mechanism for applying the optimum amount of clamping force to tube 12. Tube holder assembly 24 comprises a tube guide 80 defining a space 82 for receiving tube 12 and a locking arm 84 for releasably holding tube 12 within space 82. Tube guide 80 is generally C-shaped having a curved closed end 86 mounted on a frame sidewall 20 and an open end 88 to enable tube 12 to be placed in or removed from guide 80 when tube holder assembly 24 is in an open position. A first end 90 of locking arm 84 is pivotably coupled to a post 92 extending upwardly from base 18 adjacent tube guide 80. In some embodiments a second end 94 of locking arm 84 is configured to engage a rib 96 extending upwardly from base 18 adjacent another side of tube guide 80. More particularly, second end 94 may comprise a plurality of spaced teeth 98 which ratchedly engage a plurality of mating teeth 100 formed on rib 96. Locking arm 84 may also include a stopper block 102 located in a mid-portion thereof which blocks the open end 88 of guide 80 when locking arm 84 is in the closed position, thereby securely holding tube 12 within space 82 in a fixed position. In some embodiments stopper block 102 may be resiliently flexible to bear against tube 12 when locking arm 84 is in the closed position. In order to accommodate tubes 12 of different diameter, locking arm 84 may be adjustable to alter the position of stopper block 102 and hence the effective size of space 82 and clamping force applied to tube 12. As in the embodiment of FIGS. 1-30, guide 80 may be centered on a transverse centreline of apparatus 10 (FIG. 35) in alignment with the longitudinal axis of incision 14.

In use, apparatus 10 of FIG. 31-47 is deployed in a manner generally similar to apparatus 10 of FIGS. 1-34. Ordinarily the first step is to move tube 12 to an end of incision 14 and position apparatus 10 at a location to receive tube 12 within guide 80. Guide 80 may then be closed by adjusting locking arm 84 to a position wherein mating teeth 98, 100 are ratchedly engaged. In this closed position stopper block 98 bears against a surface of tube 12 to hold it securely in place within guide space 82. If necessary, locking arm 84 can be released and tube 12 can be relocated or repositioned as required during the treatment period. After any tube position adjustment, locking arm 84 can then be reset to once again fix tube 12 in place as described above.

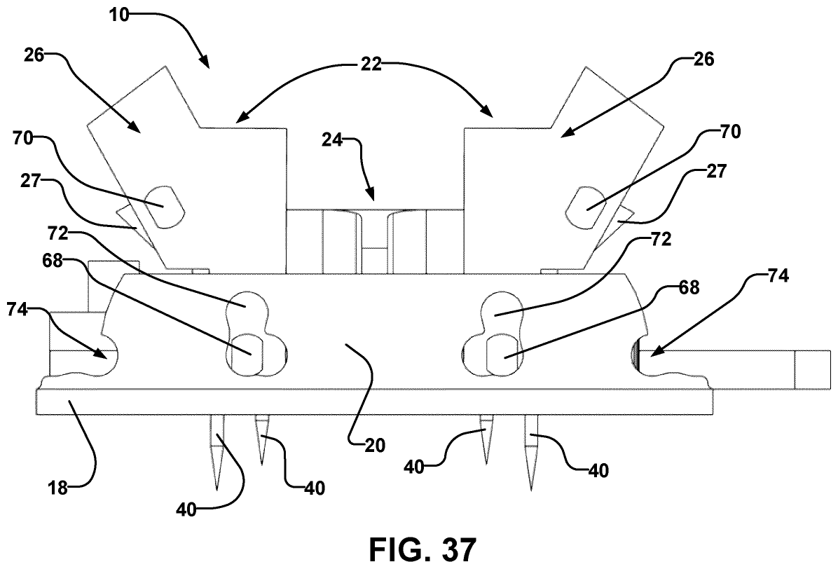

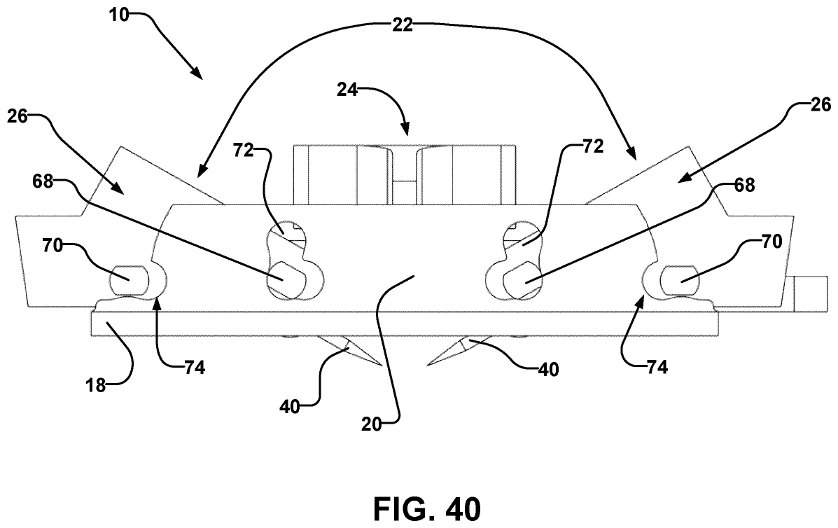

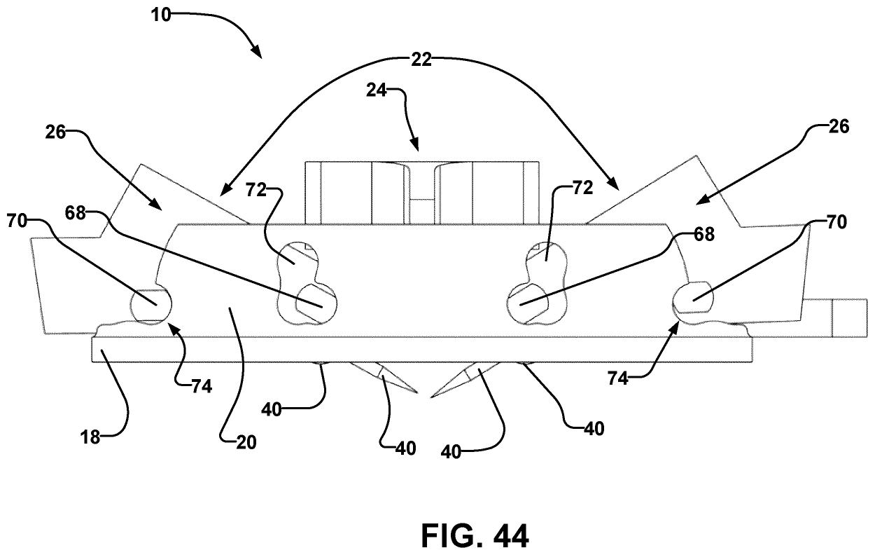

FIG. 33 is a first side view of apparatus 10 in an initial position. FIG. 34 is an enlarged second side view of apparatus 10 in an initial position (locking arm 84 is omitted in the side views for clarity). In the initial position of FIGS. 33 and 34, the free end of needles 40 and 40A may be aligned with base 18 or extend slightly below base 18 for penetration into the patient's tissue (i.e. needles 40 are not retracted within a housing of handles 26 as in the embodiment of FIGS. 1-30 described above). The first step in the needle engagement procedure is to press handles 26 vertically downwardly to move pivot pin 68 from an upper portion of generally L-shaped slot 72 to a lower, outer portion thereof (FIGS. 37-39). This causes needles 40 and 40A to further project below base 18 to penetrate to a greater depth in the patient's tissue adjacent incision 14. Needle 40A, which may be longer in length than the other needles 40, is located immediately adjacent tube 12 to more deeply engage tissue in the immediate vicinity of tube 12. The next step in the deployment procedure is to pivot handles 26 downwardly relative to fixed frame 16 about the axis of pivot pin 68 to cause eversion (lifting) of the patient's tissue adjacent incision 14 and partial closure of the incision as the everted tissue is brought together (FIGS. 40-43). At this stage of deployment, pin 70 is seated immediately adjacent slot 74 formed in an end portion of sidewall 20. In the final step of the deployment procedure handles 26 are pressed toward one another to cause inward displacement (i.e. toward incision 14) in a generally horizontal plane, assuming the patient is in a supine position. This causes pivot pin 68 to move in generally L-shaped slot 72 from a lower, outer portion thereof to a lower, inner portion thereof (FIGS. 44-47). At the same time pin 70 is fully seated within slot 74 to releasably secure handles 26 in place. In this fully deployed position locking tab 27 is received in slot 19 formed in base 18 to releasably lock each handle 26 in place. This ensures that tube 12 is stably supported in the closed position and incision 14 remains sealed. In this configuration the everted tissue is brought into contact along the length of incision 14 to close and seal incision 14 as described above.

The embodiment of FIGS. 31-47 is not adapted for post-closure adjustment of handles 26 in a manner similar to the embodiment of FIGS. 1-30. However, as will be apparent to a person skilled in the art, further embodiments can be envisaged permitting horizontal, vertical or other adjustment of the position of handles 26 in the fully deployed position. As in the embodiment of FIGS. 1-30 described above, apparatus 10 may be removed from a patient by reversing the deployment steps described above. In some embodiments locking tab 27 may be released from slot 19 by inserting a tool into cut-away opening 29 and manually lifting tab 27. As will be apparent to a person skilled in the art, many other means for releasably engaging and disengaging a handle locking mechanism may be envisaged.

As in the embodiment of FIGS. 1-30, apparatus 10 of FIGS. 31-47 permits visualization of incision 14 both during and after deployment. Further, the embodiment of FIGS. 31-47 also has a relatively low profile in the fully deployed configuration.

Although embodiments of apparatus 10 have been described herein for holding a chest tube 12, a person skilled in the art will appreciate that apparatus 10 could be used to support other types of tubes, catheters or other medical or other instruments insertable through an incision, wound or other body opening. Further, although deployment of apparatus 10 has been described herein with reference to closure of a surgically formed incision 14, apparatus 10 could be employed for closure of other bodily wounds or temporary openings without the need for sutures, adhesives and/or other medical instruments. In other embodiments, apparatus 10 could optionally be used in conjunction with other surgical materials and/or devices, such as adhesives for maintaining apparatus 10 at the optimum location on a patient's body during deployment.

In other embodiments of the invention apparatus 10 could be adapted for closing an incision, wound or other opening without concurrently holding a tube 12 other medical instrument. That is, in some medical applications and embodiments tube holder assembly 24 could be omitted entirely. For example, apparatus 10 could be used to close an incision opening made in tissue for cosmetic or other purposes unrelated to insertion of a tube 14.

As will be understood by a person skilled in the art, a patient will ordinarily be in a supine position when a chest tube is inserted and apparatus 10 is deployed. However, depending upon the patient and the medical procedure or application required, the patient may be in a different position or orientation during deployment of apparatus 10. Further, after apparatus 10 has been fully deployed as described above, the patient may move between supine, sitting, standing and or other positions with apparatus 10 fixed in place.

### Interpretation of Terms

Unless the context clearly requires otherwise, throughout the description and the
“comprise”, “comprising”, and the like are to be construed in an inclusive sense, as opposed to an exclusive or exhaustive sense; that is to say, in the sense of “including, but not limited to”;
“connected”, “coupled”, or any variant thereof, means any connection or coupling, either direct or indirect, between two or more elements; the coupling or connection between the elements can be physical, logical, or a combination thereof; elements which are integrally formed may be considered to be connected or coupled;
“herein”, “above”, “below”, and words of similar import, when used to describe this specification, shall refer to this specification as a whole, and not to any particular portions of this specification;
“or”, in reference to a list of two or more items, covers all of the following interpretations of the word: any of the items in the list, all of the items in the list, and any combination of the items in the list;
the singular forms “a”, “an”, and “the” also include the meaning of any appropriate plural forms.

Words that indicate directions such as “vertical”, “transverse”, “horizontal”, “upward”, “downward”, “forward”, “backward”, “inward”, “outward”, “vertical”, “transverse”, “left”, “right”, “front”, “back”, “top”, “bottom”, “below”, “above”, “under”, and the like, used in this description and any accompanying claims (where present), depend on the specific orientation of the apparatus described and illustrated. The subject matter described herein may assume various alternative orientations. Accordingly, these directional terms are not strictly defined and should not be interpreted narrowly.

Although the operations of the method(s) herein are shown and described in a particular order, the order of the operations of each method may be altered so that certain operations may be performed in an inverse order or so that certain operation may be performed, at least in part, concurrently with other operations. In another embodiment, instructions or sub-operations of distinct operations may be in an intermittent and/or alternating manner.

Where a component (e.g. a bolt, gear, etc.) is referred to above, unless otherwise indicated, reference to that component (including a reference to a “means”) should be interpreted as including as equivalents of that component any component which performs the function of the described component (i.e. that is functionally equivalent), including components which are not structurally equivalent to the disclosed structure which performs the function in the illustrated exemplary embodiments of the invention.

Specific examples of systems, methods and apparatus have been described herein for purposes of illustration. These are only examples. The technology provided herein can be applied to systems other than the example systems described herein. Many alterations, modifications, additions, omissions, and permutations are possible within the practice of this invention. This invention includes variations on described embodiments that would be apparent to the skilled addressee, including variations obtained by: replacing features, elements and/or acts with equivalent features, elements and/or acts; mixing and matching of features, elements and/or acts from different embodiments; combining features, elements and/or acts from embodiments as described herein with features, elements and/or acts of other technology; and/or omitting combining features, elements and/or acts from described embodiments.

Various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventors also contemplate embodiments which combine features A and B (unless the description states otherwise or features A and B are fundamentally incompatible).

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are consistent with the broadest interpretation of the specification as a whole.

1. An apparatus for closing an incision formed in a patient and for releasably securing a tube passing through the incision comprising:
(a) a frame having a first side and a second side;
(b) a handle assembly pivotably coupled to the frame, the assembly comprising a pair of handles at least partially disposed between the first side and the second side, the pair of handles comprising a first handle movable about a first pivot axis and a second handle movable about a second pivot axis spaced apart from the first pivot axis, wherein a viewing space is defined between the first handle and the second handle for viewing the incision;
(c) a needle array positionable at a first end of each of the first handle and the second handle for penetrating tissue of the patient adjacent to the incision; and
(d) a tube holder located adjacent one of the first side and the second side for securing the tube.

2. The apparatus as defined in claim 1, wherein each of the first handle and the second handle is movable relative to the frame between a disengaged position wherein the needle array does not penetrate the tissue of the patient and an engaged position wherein the needle array penetrates the tissue of the patient.

3. The apparatus of claim 2, wherein each of the first handle and the second handle comprises a housing and a needle cartridge comprising the needle array, wherein the cartridge is movable in the housing between the disengaged and the engaged positions.

4. The apparatus of claim 3, wherein the needle array is retracted within the housing in the disengaged position.

5. The apparatus as defined in claim 1, wherein the incision has a length between a first end and a second end thereof, and wherein the apparatus is positionable on the patient with the incision extending between the handles within the viewing space.

6. The apparatus as defined in claim 1, wherein the handle assembly is movable between an inserted position wherein the needle array of each of the handles penetrates the tissue of the patient and an everted position wherein the tissue of the patient engaged by the handles is everted.

7. The apparatus as defined in claim 6, wherein the needle array of the first handle engages the tissue on a first side of the incision and the needle array of the second handle engages the tissue on a second side of the incision, wherein the handle assembly is configured for bringing everted tissue on the first side and the second side together to close the incision.

8. The apparatus as defined in claim 6, wherein the handle assembly is pivotably movable between the inserted position and the everted position.

9. The apparatus as defined in claim 6, wherein the distance between the first handle and the second handle is adjustable in the everted position to alter the distance between the tissue on the first side and the second side of the incision.

10. The apparatus as defined in claim 6, wherein the handle assembly is releasably lockable in closed position wherein the everted tissue is in contact and the incision is closed.

11. The apparatus as defined in claim 6, wherein the handle assembly is releasably lockable in the everted position.

12. The apparatus as defined in claim 6, wherein the distance between the first handle and the second handle is adjustable in the everted position to adjust the compressive force applied to the tissue on either side of the incision in contact when the incision is closed.

13. The apparatus as defined in claim 5, wherein the tube holder is positionable overlying the first end or the second end of the incision.

14. The apparatus as defined in claim 1, wherein the tube holder is configured to securely receive a chest tube extending through an end portion of the incision.

15. The apparatus as defined in claim 1, wherein the viewing space is generally aligned with a transverse centreline of the frame extending between the first side and the second side.

16. The apparatus as defined in claim 15, wherein the tube guide is a generally U-shaped holder having a central portion aligned with the transverse centreline.

17. The apparatus as defined in claim 15, wherein the apparatus is positionable relative to the incision such that the length of the incision is generally aligned with the transverse centreline of the frame.

18. The apparatus as defined in claim 1, wherein the needle array comprises a plurality of spaced-apart needles.

19. The apparatus as defined in claim 18, wherein at least some of the needles are oriented at a non-vertical angle differing from the other needles.

20. The apparatus as defined in claim 1, wherein each of the first handle and the second handle comprises pivot pins projecting laterally from portions thereof in alignment with the pivot axis and second pivot axis respectively, each of the pivot pins having an end portion received in a guide slot of the apparatus, wherein each pivot pin moves within a corresponding guide slot when the handles are moved relative to the frame.

21. The apparatus of claim 20, wherein each guide slot is formed in the first side or the second side of the frame.

22. The apparatus of claim 20, wherein each guide slot is formed in a handle carriage movable relative to the first side and/or second side of the frame.

23. The apparatus of claim 21, wherein a proximal guide slot located proximal to the tube holder is sized and/or shaped differently from a distal guide slot located distal from the tube holder.

24. The apparatus as defined in claim 20, wherein each of the guide slots is generally L-shaped.

25. The apparatus as defined in claim 6, wherein each of the first handle and the second handle comprises at least one laterally projecting pivot pin movable in a slot formed in one of the first or second sides when the first handle and the second handle are adjusted between the inserted and the everted positions.

26. The apparatus as defined in claim 1, comprising a locking arm for releasably locking a tube within the tube holder.

27. The apparatus as defined in claim 1, wherein the viewing space is defined between inner surfaces of the first handle and the second handle, wherein the size of the space varies between an initial position and a deployed position, wherein, in the initial position, the distance between the inner surfaces of the first handle and the second handle diverges from a maximum distance at the first side of the frame proximal to the tube holder to a minimum distance at the second side of the frame distal from the tube holder, wherein the extent of travel of each first handle and second handle varies over the length thereof when the handles are moved between the initial and deployed positions.

28. The apparatus as defined in claim 27, wherein, in the deployed position, the inner surfaces of the first handle and the second handle extend substantially co-planar with a transverse centreline of the frame and the distance between the inner surfaces of the handles is substantially constant.

29. The apparatus as defined in claim 1, wherein the height of the apparatus in a deployed configuration is less than approximately 3 cm.

30. The apparatus as defined in claim 1, wherein the needle array is removably connectable to a respective one of the handles.

31. The apparatus as defined in claim 3, wherein the apparatus comprises a plurality of needle cartridges, wherein at least some each one of the cartridges is removably connectable to one of the handles, and wherein at least some of the cartridges comprise a needle configuration differing from other of the cartridges.

32. A method of closing an incision formed in a patient, the incision having a length extending between a first end and a second end thereof, the method comprising:
(a) providing an apparatus as defined in claim 1;
(b) positioning the apparatus overlying the incision such that the incision is aligned between the handles;
(c) penetrating the needle array of each of the handles into the tissue of the patient adjacent to the incision;
(d) moving the handles relative to the frame to evert the tissue adjacent to the incision; and
(e) bringing everted tissue on either side of the incision into contact to close the incision.

33. The method as defined in claim 32, further comprising, prior to moving the handles relative to the frame, positioning a tube at one end of the incision and releasably securing the tube within the tube holder.

34. The method as defined in claim 32, further comprising locking the handles relative to the frame to securely maintain the everted tissue on either side of the incision in contact.

35. The method as defined in claim 32, further comprising adjusting the distance between the handles when the tissue is everted to adjust the compressive force applied to the tissue when the incision is closed.

36. A method of inserting a chest tube in a patient comprising:
(a) making an incision in a chest wall of the patient;
(b) inserting the chest tube through the incision and placing the chest tube at one end of the incision;
(c) providing an apparatus as defined in claim 1;
(d) positioning the apparatus such that the incision is aligned between the handles and the chest tube is received within the tube holder;
(e) releasably securing the chest tube in the tube holder;
(f) moving the needle array relative to the frame from a disengaged position to an engaged position to cause the needles to penetrate tissue adjacent the incision; and
(g) moving the handles relative to the frame in the engaged position to evert the tissue adjacent to the incision and bring the everted tissue in contact to close the incision.

37. The method as defined in claim 36, wherein the handles are moved toward one another in the engaged position to close the incision.

38. The method as defined in claim 36, wherein the handles are releasably lockable in a closed position maintaining the everted tissue in contact.

39. The method as defined in claim 38, comprising adjusting the distance between the handles in the closed position to vary the pressure applied to the everted tissue.

40. A method of closing an incision opening formed in tissue of a patient comprising:
(a) providing an incision closure apparatus comprising a frame and a handle assembly movably coupled to the frame, wherein the handle assembly comprises a first handle comprising a first needle array and a second handle spaced-apart from the first handle and comprising a second needle array;
(b) positioning the incision closure apparatus on the patient with the incision opening disposed between the first and second handles;
(c) inserting the first needle array into the tissue adjacent a first side of the incision opening and inserting the second needle array into the tissue adjacent a second side of the incision opening;
(d) moving the handles relative to the frame to evert the tissue engaged by the first and second needle arrays; and
(e) closing the incision opening by moving the handles toward one another to bring the everted tissue in contact in a closed position.

41. The method as defined in claim 40, comprising releasably locking the first and second handles relative to the frame in the closed position.

42. The method as defined in claim 40, wherein the first needle array is detachably connectable to the first handle and the second needle array is detachably connectable to the second handle.

43. The method as defined in claim 40, wherein each of first and second handles comprises a housing and where each of the first and second needle arrays is located on a cartridge movable in the housing.

44. The method as defined in claim 43, wherein the cartridge is adjustable between a disengaged position wherein the corresponding first or second needle array is retracted within the housing and an engaged position wherein the corresponding first or second needle array extends from the housing for penetrating the tissue.

45. The method as defined in claim 40, wherein each of the first and second needle arrays comprises a plurality of spaced-apart needles.

46. The method as defined in claim 45, wherein each of the first and second needle arrays comprises at least one needle disposed in an orientation for insertion in the tissue at an angle differing from the angle of insertion of a plurality of other of the needles.

47. The method as defined in claim 40, wherein the handle assembly is pivotably coupled to the frame.

48. The method as defined in claim 40, wherein each of the first and second handles is pivotably coupled to the frame.

49. The method as defined in claim 40, wherein, during the inserting, each of the first and second needle arrays is movable in a first plane generally perpendicular to the tissue to cause the needle array to penetrate the tissue and wherein, during at least part of the closing, each of the first and second needle arrays is movable in a second plane generally perpendicular to the first plane.

50. The method as defined in claim 49, wherein, when the patient is in a supine position, the first plane is a generally vertical plane and the second plane is a generally horizontal plane.

51. The method as defined in claim 40, comprising adjusting the spacing between the handles in the closed position to vary forces applied to the everted tissue.

52. The method as defined in claim 41, comprising adjusting the spacing between the handles after the locking.

53. The method as defined in claim 40, comprising inserting a tube through the incision opening and releasably coupling the apparatus to the tube.

54. The method as defined in claim 53, wherein the tube is positioned at one end of the incision opening.

55. The method as defined in claim 53, wherein the coupling is performed prior to the inserting.

56. The method as defined in claim 53, wherein the apparatus comprises a tube holder connected to the frame and wherein the coupling comprises placing the tube in the tube holder.

57. The method as defined in claim 56, wherein the apparatus comprises a transverse centreline extending between the first and second handles and alignable with the incision opening during the positioning and wherein the tube holder is aligned with the transverse centreline.

58. The method as defined in claim 40, wherein a width of the opening varies along a length thereof between a first end and a second end of the incision and wherein, during the moving the handles relative to the frame, a first portion of each of the handles adjacent the first end of the incision travels a different distance than a second portion of each of the handles adjacent the second end of the incision.

59. The method as defined in claim 40, wherein the moving comprises rotating the handles relative to the frame.

60. An incision closure apparatus for closing an opening formed in tissue of a patient comprising:
(a) a frame;
(b) a handle assembly movably coupled to the frame, wherein the handle assembly comprises a first handle comprising a first needle array and a second handle spaced-apart from the first handle and comprising a second needle array, wherein each of the first and second needle arrays is movable in a first plane generally perpendicular to the tissue to cause the needle arrays to penetrate the tissue and a second plane generally perpendicular to the first plane to bring penetrated tissue together to close the incision.

61. The apparatus as defined in claim 60, wherein the handle assembly is pivotably coupled to the frame, wherein each of the first handle and the second handle is pivotable after the needle arrays have penetrated the tissue to evert the tissue.

62. (canceled)

63. (canceled)

64. (canceled)

65. An apparatus for closing an incision formed in a patient and for releasably securing a tube passing through the incision comprising:
(a) a frame having a first side and a second side;
(b) a handle assembly pivotably coupled to the frame, the assembly comprising a pair of handles at least partially disposed between the first side and the second side, the pair of handles comprising a first handle and a second handle spaced-apart from the first handle to define a viewing space therebetween;
(c) a needle array positionable at a first end of each of the first handle and the second handle for penetrating tissue of the patient adjacent to the incision; and
(d) a tube holder located adjacent one of the first side and the second side for securing the tube,
wherein each of the first handle and the second handle comprises a housing and a needle cartridge comprising the needle array, wherein the cartridge is movable relative to the housing between disengaged and the engaged positions.

66. An apparatus for closing an incision formed in a patient and for releasably securing a tube passing through the incision comprising:
(a) a frame having a first side and a second side;
(b) a handle assembly pivotably coupled to the frame, the assembly comprising a pair of handles at least partially disposed between the first side and the second side, the pair of handles comprising a first handle and a second handle spaced-apart from the first handle to define a viewing space therebetween;
(c) a needle array positionable at a first end of each of the first handle and the second handle for penetrating tissue of the patient adjacent to the incision; and
(d) a tube holder located adjacent one of the first side and the second side for securing the tube;
wherein each of the first handle and the second handle comprises pivot pins projecting laterally from portions thereof, each of the pivot pins having an end portion received in a guide slot of the apparatus configured to permit pivotable movement of the respective first handle and second handle relative to the frame.
